(19) United States
(12) Patent Application Publication
(10) Pub. No.: US 2003/0064429 A1
(43) Pub. Date: Apr. 3, 2003

(54) Efficient methods for the preparation of rhinovirus protease inhibitors, key intermediates and a continuous membrane reactor useful for preparing the same

(75) Inventors: Junhua Tao, San Diego, CA (US); Srinivasan Babu, San Diego, CA (US); Raymond Dagnino JR., El Cajon, CA (US); Qingping Tian, San Diego, CA (US); Travis Paul Remarchuk, Irvine, CA (US); Kevin Scott McGee, San Diego, CA (US); Naresh K. Nayyar, San Diego, CA (US); Terence Jarold Moran, San Diego, CA (US)

Correspondence Address: SHANKS & HERBERT, 1033 N. FAIRFAX STREET, SUITE 306, ALEXANDRIA, VA, 22314, US

(73) Assignee: Agouron Pharmaceuticals, Inc.

(21) Appl. No.: 10/201,944

(22) Filed: Jul. 25, 2002

Related U.S. Application Data
(62) Division of application No. 09/643,865, filed on Aug. 23, 2000, now abandoned.
(60) Provisional application No. 60/150,365, filed on Aug. 24, 1999.
(60) Provisional application No. 60/150,358, filed on Aug. 24, 1999.

(51) Int. Cl. C12P 33/02; C12N 9/48; C12N 9/50

(52) U.S. Cl. 435/61; 435/212; 435/219

(57) ABSTRACT

Efficient synthetic routes for the preparation of rhinovirus protease inhibitors of formula I, key intermediates useful in those synthetic routes, as well as a continuous membrane reactor useful for those synthetic routes. These compounds of formula I, as well as pharmaceutical compositions that contain these compounds, are suitable for treating patients or hosts infected with one or more picornaviruses.

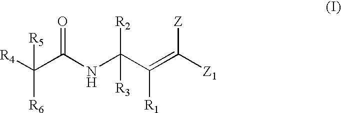

### RELATED APPLICATION DATA

This application relates to U.S. Provisional Patent Application Serial No. 60/150,365, filed on Aug. 24, 1999.

This application also relates to a U.S. Provisional Patent Application No. 60/150,358 (Attorney Docket No.: 0125.0028) entitled “Efficient Synthetic Routes For The Preparation Of Rhinovirus Protease Inhibitors And Key Intermediates” having named as inventors: Q. Tian, N. Nayyar, S. Babu, J. Tao, T. Moran, R. Dagnino, Jr., T. Remarchuk, M. Melnick, L. Mitchell, Jr., and S. Bender. This aforementioned application also relates to synthetic routes for the preparation of rhinovirus protease inhibitors and key intermediates for use therein.

The above-referenced applications are relied upon and are incorporated herein by reference.

### TECHNICAL FIELD AND INDUSTRIAL APPLICABILITY

The present invention relates to an improved process for the preparation of ethyl-3-{(5′-methylisoxazole-3′-carbonyl)-L-ValΨ(COCH₂)-L-(4-F-Phe)-L-((S)-Pyrrol-Ala)}-E-propanoate, its analogs and of pharmaceutically acceptable salts thereof. The present invention also includes a novel group of intermediate compounds to be used in the above process. Additionally, the present invention includes a continuous membrane reactor useful for use with the processes of the present invention.

### BACKGROUND OF THE INVENTION

Picornaviruses are a family of tiny non-enveloped positive-stranded RNA-containing viruses that infect humans and other animals. These viruses include the human rhinoviruses, human polioviruses, human coxsackieviruses, human echoviruses, human and bovine enteroviruses, encephalomyocarditis viruses, meningitis viruses, foot and mouth viruses, hepatitis A virus, and others. The human rhinoviruses are a major cause of the common cold.

Proteolytic 3C enzymes are required for the natural maturation of the picornaviruses. Thus, inhibiting the activity of these proteolytic 3C enzymes should represent an important and useful approach for the treatment and cure of viral infections of this nature, including the common cold.

Some small-molecule inhibitors of the enzymatic activity of picornaviral 3C protease (i.e., antipicornaviral compounds) have been recently discovered. See, for example: U.S. patent application Ser. No. 08/850,398, filed May 2, 1997, by Webber et al.; U.S. patent application Ser. No. 08/991,282, filed Dec. 16, 1997, by Dragovich et al.; and U.S. patent application Ser. No. 08/991,739, filed Dec. 16, 1997, by Webber et al. These U.S. patent applications, the disclosures of which are incorporated herein by reference, describe certain antipicornaviral compounds and methods for their synthesis.

More recently, an especially potent group of antipicornaviral agents have been discovered as set forth in U.S. patent application Ser. No. 60/098,354, (the '354 application) filed Aug. 28, 1998, by Dragovich et al., which is herein incorporated by reference. This application discloses, inter alias, a group of antipicornaviral agents of general formula I. A particularly promising compound, AG7088, falling within the scope of this group, exhibits excellent antiviral properties against a plethora of Rhinoviral serotypes and is currently in human clinical trials. The '354 application also discloses methods and intermediates useful for synthesizing these compounds. For example, General Method V therein discloses a general method for synthesizing the compounds of formula I involving subjecting a carboxylic acid of general formula BB to an amide-forming reaction with an amine of general formula P to provide a final product CC, as shown below.

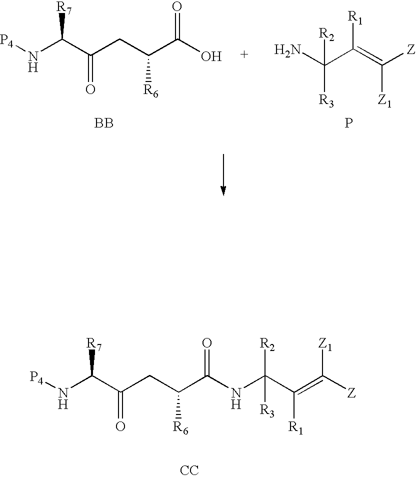

The '354 application discloses methods for synthesizing the intermediates of general formulae BB and P, and teaches methods for carrying out the amide-forming reaction referred to above. Thus, the '354 application teaches suitable methods for synthesizing the compounds of general formula I from a carboxylic acid BB (within the scope of the compounds of general formula II referred to below) and the compounds of general formula P (the same as the compounds of general formula III referred to below.)

Similarly, two recent publications by Dragovich et al. disclose antipicornavirus agents and suitable synthetic methods for their synthesis. See Structure-Based Design, Synthesis, and Biological Evaluation of Irreversible Human Rhinovirus 3C Proteases Inhibitors. 3. StructureActivity Studies of Ketomethylene-Containing Peptidomimetics, Dragovich et al., Journal of Medicinal Chemistry, ASAP, 1999; and Structure-Based Design, Synthesis, and Biological Evaluation of Irreversible Human Rhinovirus 3C Proteases Inhibitors. 4. Incorporation of P₁ Lactam Moieties as L-Glutamine Replacements, Dragovich et al., Journal of Medicinal Chemistry, ASAP, 1999. These aforementioned articles are herein incorporated by reference in their entirety.

However, there is still a desire to discover improved, more efficient, processes and novel intermediates for use in the syntheses of the compounds of the group of antipicornaviral agents. In particular, there is a need for improved methods for synthesizing the compounds of general formulae I, II and III.

The process of the present invention involves an enzymatic reduction step. Due to the expense of certain catalysts, including enzymatic catalysts, there has been a need to recycle these expensive catalysts. This has been done, inter alias, by use of a continuous membrane reactor. The development of continuous membrane reactors has made the use of these expensive catalysts economically feasible in the preparation of compounds. However, up until the present invention, continuous membrane reactors have been expensive and lacked the versatility to significantly vary the scale of the catalytic reaction. Specifically, known continuous membrane reactors employ hollow fiber filter reactors, in which a majority of the volume of the reagent(s) and enzyme(s) is present, where the majority of the enzymatic reaction occurs. Accordingly, to vary the scale of the reaction, a different hollow fiber filter reactor of appropriate size must be employed. See, for example, E. Schmidt et al., Journal of Biotechnology, 24 (1992) 315-327, which discloses a continuous membrane reactor. The aforementioned article is herein incorporated by reference. Further, due to the expense of hollow fiber filter reactors, the known continuous membrane reactors tend to be expensive. Thus, there is a need for a more economical and versatile continuous membrane reactor.

### SUMMARY OF THE INVENTION

The present invention relates to the discovery of a cost effective and efficient process for the preparation of the antipicornaviral agents of formula I, such as compound AG7088, as well as intermediates which are useful in that synthesis.

The antipicornaviral agents of formula I comprise:

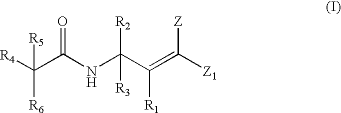

wherein R₁ is H, F, an alkyl group, OH, SH, or an O-alkyl group;

R₂ and R₃ are each independently H;

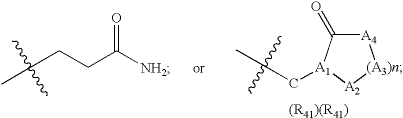

where n is an integer from 0 to 5, A₁ is CH or N, A₂ and each A₃ are independently selected from C(R₄₁)(R₄₁), N(R₄₁), S, S(O), S(O)₂, and O, and A₄ is NH or NR₄₁, where each R₄₁ is independently H or lower alkyl, provided that no more than two heteroatoms occur consecutively in the above-depicted ring formed by A₁, A₂, (A₃)ₙ, A₄ and C═O, and at least one of R₂ and R₃ is

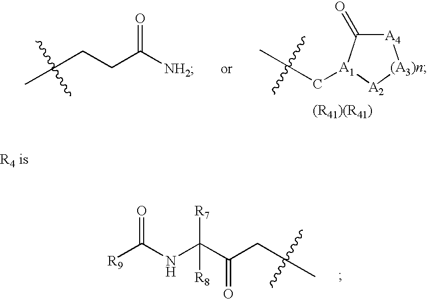

R₅ and R₆ are each independently H, F, an alkyl group, a cycloalkyl group, a heterocycloalkyl group, an aryl group, or a heteroaryl group;

R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈, where R₁₇, R₁₈, and R₁₉ are each independently H, an alkyl group, a cycloalkyl group, a heterocycloalkyl group, an aryl group, a heteroaryl group, or an acyl group, provided that at least one of R₇ and R₈ is an alkyl group, an aryl group, a heteroaryl group, —OR₁₇, —SR₁₇, —NR₁₇R₁₈, —NR₁₉NR₁₇R₁₈, or —NR₁₇OR₁₈;

R₉ is a five-membered heterocycle having from one to three heteroatoms selected from O, N, and S; and

Z and Z₁ are each independently H, F, an alkyl group, a cycloalkyl group, a heterocycloalkyl group, an aryl group, a heteroaryl group, —C(O)R₂₁, —CO₂R₂₁, CN, —C(O)NR₂₁R₂₂, —C(O)NR₂₁OR₂₂, —C(S)R₂₁, —C(S)NR₂₁R₂₂, —NO₂, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), —PO(NR₂₁R₂₂)(NR₂₃R₂₄), —C(O)NR₂₁NR₂₂R₂₃, or —C(S)NR₂₁NR₂₂R₂₃, where R₂₁, R₂₂, R₂₃, and R₂₄ are each independently H, an alkyl group, a cycloalkyl group, a heterocycloalkyl group, an aryl group, a heteroaryl group, an acyl group, or a thioacyl group, or where any of two of R₂₁, R₂₂, R₂₃, and R₂₄, together with the atom(s) to which they are bonded, form a heterocycloalkyl group, provided that Z and Z₁ are not both H;

or Z₁ and R₁, together with the atoms to which they are bonded, form a cycloalkyl or heterocycloalkyl group, where Z₁ and R₁ are as defined above except for moieties that cannot form the cycloalkyl or heterocycloalkyl group;

or Z and Z₁, together with the atoms to which they are bonded, form a cycloalkyl or heterocycloalkyl group, where Z and Z₁ are as defined above except for moieties that cannot form the cycloalkyl or heterocycloalkyl group.

As discussed above, these antipicornaviral agents of formula I may be synthesized by subjecting a compound of general formula II together with a compound of general formula III to a suitable amide-forming reaction. The process of the present invention provides a more cost effective and efficient method of synthesizing the compounds of formula I from the compounds of formulae II and III.

The process of the present invention also provides more cost effective and efficient methods of synthesizing the compounds of formula II, thus, providing an improved overall method for synthesizing the antipicornaviral agents of formula I.

Additionally, the present invention provides novel intermediates for use in the processes of the present invention, and novel processes for the preparation of those novel intermediates.

The present invention also relates to a continuous membrane reactor that may be used in the processes of the present invention.

These objects, advantages and features of the present invention will be more fully understood and appreciated by reference to the written specification.

### DETAILED DESCRIPTION OF A PREFERRED EMBODIMENT OF THE INVENTION

As used in the present application, the following definitions apply:

In accordance with a convention used in the art,

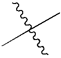

is used in structural formulas herein to depict the bond that is the point of attachment of the moiety or substituent to the core or backbone structure.

Where chiral carbons are included in chemical structures, unless a particular orientation is depicted, both sterioisomeric forms are intended to be encompassed.

An “alkyl group” is intended to mean a straight- or branched chain monovalent radical of saturated and/or unsaturated carbon atoms and hydrogen atoms, such as methyl (Me), ethyl (Et), propyl, isopropyl, butyl (Bu), isobutyl, t-butyl (t-Bu), ethenyl, pentenyl, butenyl, propenyl, ethynyl, butynyl, propynyl, pentynyl, hexynyl, and the like, which may be unsubstituted (i.e., containing only carbon and hydrogen) or substituted by one or more suitable sustituents as defined below (e.g., one or more halogens, such as F, Cl, Br, or I, with F and Cl being preferred). A “lower alkyl group” is intended to mean an alkyl group having from 1 to 4 carbon atoms in its chain.

A “cycloalkyl group” is intended to mean a non-aromatic monovalent monocyclic, bicyclic, or tricyclic radical containing 3, 4, 5, 6, 7, 8, 9, 10, 11, 12, 13, or 14 carbon ring atoms, each of which may be saturated or unsaturated, and which may be unsubstituted or substituted by one or more suitable substituents as defined below, and to which may be fused one or more heterocycloalkyl groups, aryl groups, or heteroaryl groups, which themselves may be unsubstituted or substituted by one or more substituents. Illustrative examples of cycloalkyl groups include following moieties:

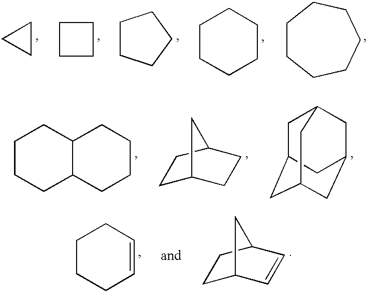

A “heterocycloalky group” is intended to mean a non-aromatic monovalent monocyclic, bicyclic, or tricyclic radical, which is saturated or unsaturated, containing 3, 4, 5, 6, 7, 8, 9, 10, 11, 12, 13, 14, 15, 16, 17, or 18 ring atoms, which includes 1, 2, 3, 4, or 5 heteroatoms selected nitrogen, oxygen, and sulfur, where the radical is unsubstituted or substituted by one or more suitable substituents as defined below, and to which may be fused one or more cycloalkyl groups, aryl groups, or heteroaryl groups, which themselves may be unsubstituted, or substituted by one or more suitable substituents. Illustrative examples of heterocycloalkyl groups include the following moieties:

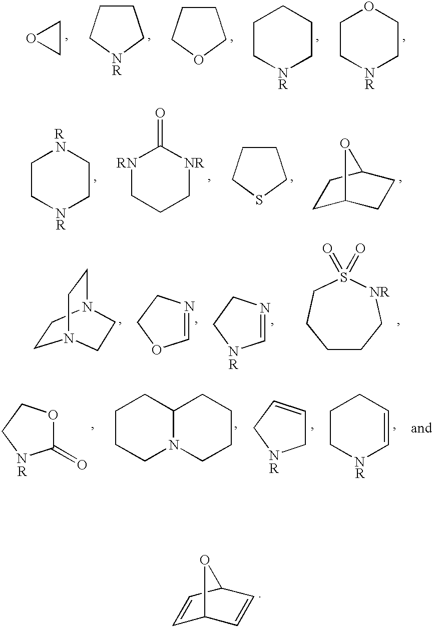

An “aryl group” is intended to mean an aromatic monovalent monocyclic, bicyclic, or tricyclic radical containing 6, 10, 14,, or 18 carbon ring atoms, which may be unsubstituted or substituted by one or more suitable substituents as defined below, and to which may be fused one or more cycloalkyl groups, heterocycloalkyl groups, or heteroaryl groups, which themselves may be unsubstituted or substituted by one or more suitable substituents. Thus, the term “aryl group” includes a benzyl group (Bzl). Illustrative examples of aryl groups include the following moieties:

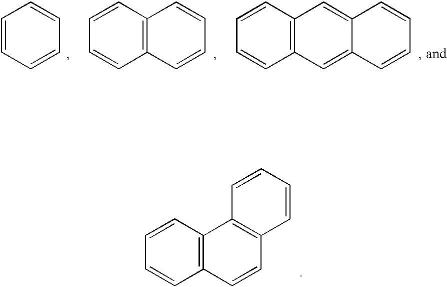

A “heteroaryl group” is intended to mean an aromatic monovalent monocyclic, bicyclic, or tricyclic radical containing 4, 5, 6, 7, 8, 9, 10, 11, 12, 13, 14, 15, 16, 17, or 18 ring atoms, including 1, 2, 3, 4, or 5 heteroatoms selected from nitrogen, oxygen, and sulfur, which may be unsubstituted or substituted by one or more suitable substituents as defined below, and to which may be fused one or more cycloalkyl groups, heterocycloalkyl groups, or aryl groups, which themselves may be unsubstituted or substituted by one or more suitable substituents. Illustrative examples of heteroaryl groups include the following moieties:

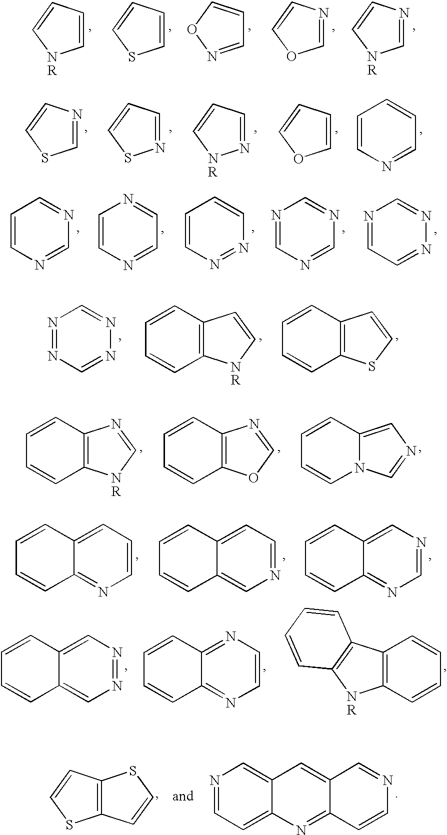

A “heterocycle” is intended to mean a heteroaryl or heterocycloalkyl group (each of which, as defined above, are optionally substituted).

An “acyl group” is intended to mean a —C(O)—R radical, where R is a substituent as defined below.

A “thioacyl group” is intended to mean a —C(S)—R radical, where R is a substituent as defined below.

A “sulfonyl group” is intended to mean a —SO₂R radical, where R is a substituent as defined below.

A “hydroxy group” is intended to mean the radical —OH.

An “amino group” is intended to mean the radical —NH₂.

An “alkylamino group” is intended to mean the radical —NHRₐ, where Rₐ is an alkyl group.

A “dialkylamino group” is intended to mean the radical —NRₐR_{b}, where Rₐ and R_{b} are each independently an alkyl group.

An “alkoxy group” is intended to mean the radical —ORₐ, where Rₐ, is an alkyl group. Exemplary alkoxy groups include methoxy, ethoxy, propoxy, and the like.

An “alkoxycarbonyl group” is intended to mean the radical —C(O)ORₐ, where Rₐ, is an alkyl group.

An “alkylsulfonyl group” is intended to mean the radical —SO₂Rₐ, where Rₐ, is an alkyl group.

An “alkylaminocarbonyl group” is intended to mean the radical —C(O)NHRₐ, where Rₐ is an alkyl group.

A “dialkylaminocarbonyl group” is intended to mean the radical —C(O)NRₐR_{b}, where Rₐ, and R_{b} are each independently an alkyl group.

A “mercapto group” is intended to mean the radical —SH.

An “alkylthio group” is intended to mean the radical —SRₐ, where Rₐ is an alkyl group.

A “carboxy group” is intended to mean the radical —C(O)OH.

A “carbamoyl group” is intended to mean the radical —C(O)NH₂.

An “aryloxy group” is intended to mean the radical —OR_{c}, where R_{c} is an aryl group.

A “heteroaryloxy group” is intended to mean the radical —OR_{d}, where R_{d} is a heteroaryl group.

An “arylthio group” is intended to mean the radical —SR_{c}, where R_{c} is an aryl group.

A “heteroarylthio group” is intended to mean the radical —SR_{d}, where R_{d} is a heteroaryl group.

A “leaving group” (Lv) is intended to mean any suitable group that will be displaced by a substitution reaction. One of ordinary skill in the art will know that any conjugate base of a strong acid can act as a leaving group. Illustrative examples of suitable leaving groups include, but are not limited to, —F, —Cl, —Br, alkyl chlorides, alkyl bromides, alkyl iodides, alkyl sulfonates, alkyl benzenesulfonates, alkyl p-toluenesulfonates, alkyl methanesulfonates, triflate, and any groups having a bisulfate, methyl sulfate, or sulfonate ion.

Typical protecting groups, reagents and solvents such as, but not limited to, those listed below in table 1 have the following abbreviations as used herein and in the claims. One skilled in the art would understand that the compounds listed within each group may be used interchangeably; for instance, a compound listed under “reagents and solvents” may be used as a protecting group, and so on. Further, one skilled in the art would know other possible protecting groups, reagents and solvents; these are intended to be within the scope of this invention.

TABLE 1

| | |
|---|---|
| | Protecting Groups |
| Ada | Adamantane acetyl |
| Alloc | Allyloxycarbonyl |
| Allyl | Allyl ester |
| Boc | tert-butyloxycarbonyl |
| Cbz | Benzyloxycarbonyl |
| Fmoc | Fluorenylmethyloxycarbonyl |
| OBzl | Benzyl ester |
| OEt | Ethyl ester |
| OMe | Methyl ester |
| Tos (Tosyl) | p-Toluenesulfonyl |
| Trt | Triphenylmethyl |
| | Reagents and Solvents |
| ACN | Acetonitrile |
| AcOH | Acetic acid |
| Ac.sub.2 O | Acetic acid anhydride |
| AdacOH | Adamantane acetic acid |
| AIBN | 2,2-azobisisobutyronitrile |
| Alloc-Cl | Allyloxycarbonyl chloride |
| BHT | 2,6-di-tert-butyl-4-methylphenol |
| Boc.sub.2 O | Di-tert butyl dicarbonate |
| CDI | 1,1′-carbonyldiimidazole |
| CDMT | Chlorodimethyltriazine |
| DCM | Methylene chloride |
| DIEA | Diisopropylethylamine |
| DIPEA | N,N-diisopropylethylamine |
| DMA | Dimethylacetamide |
| DMF | N,N-dimethylformamide |
| DMSO | Dimethyl sulfoxide |
| EDTA | ethylenediaminetetraacetic acid |
| Et.sub.3 N | Triethylamine |
| EtOAc | Ethyl acetate |
| FDH | formate dehydrogenase |
| FmocOSu | 9-fluorenylmethyloxy carbonyl |
| | N-hydroxysuccinimide ester |
| HATU | N-[(dimethylamino)-1H-1,2,3-triazol [4,5-b] |
| | pyridiylmethylene]-N-methylmethanaminium |
| | hexafluorophosphate N-oxide |
| HOBT | 1-Hydroxybenzotriazole |
| HF | Hydrofluoric acid |
| LDH | lactate dehydrogenase |
| LiHMDS | Lithium bistrimethylsilylamide |
| MeOH | Methanol |
| Mes (Mesyl) | Methanesulfonyl |
| MTBE | t-butyl methyl ether |
| NAD | Nicotinamide adenine dinucleotide |
| NADH | Hydrogen-peroxide oxidoreductase |
| NaHMDS | Sodium bistrimethylsilylamide |
| NMP | 1-methyl-2-pyrrolidinone |
| nin. | Ninhydrin |
| i-PrOH | Iso-propanol |
| Pip | Piperidine |
| PPL | Lipase |
| pTSA | p-toluensulfonic acid monohydrate |
| Pyr | Pyridine |
| TEA | Triethylamine |
| TET | triethylenetetraamine |
| TFA | Trifluoroacetic acid |
| THF | Tetrahydrofuran |
| Triflate (Tf) | Trifluoromethanesulfonyl |

The term “suitable organic moiety” is intended to mean any organic moiety recognizable, such as by routine testing, to those skilled in the art as not adversely affecting the inhibitory activity of the inventive compounds. Illustrative examples of suitable organic moieties include, but are not limited to, hydroxyl groups, alkyl groups, oxo groups, cycloalkyl groups, heterocycloalkyl groups, aryl groups, heteroaryl groups, acyl groups, sulfonyl groups, mercapto groups, alkylthio groups, alkoxy groups, carboxy groups, amino groups, alkylamino groups, dialkylamino groups, carbamoyl groups, arylthio groups, heteroarylthio groups, and the like.

The term “substituent” or suitable substituent” is intended to mean any suitable substituent that may be recognized or selected, such as through routine testing, by those skilled in the art. Illustrative examples of suitable substituents include hydroxy groups, halogens, oxo groups, alkyl groups, acyl groups, sulfonyl groups, mercapto groups, alkylthio groups, alkyloxy groups, cycloalkyl groups, heterocycloalkyl groups, aryl groups, heteroaryl groups, carboxy groups, amino groups, alkylamino groups, dialkylamino groups, carbamoyl groups, aryloxy groups, heteroaryloxy groups, arylthio groups, heteroarylthio groups, and the like.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

A “prodrug” is intended to mean a compound that is converted under physiological conditions or by solvolysis or metabolically to a specified compound that is pharmaceutically active.

A “pharmaceutically active metabolite” is intended to mean a pharmacologically active product produced through metabolism in the body of a specified compound.

A “solvate” is intended to mean a pharmaceutically acceptable solvate form of a specified compound that retains the biological effectiveness of such compound. Examples of solvates include compounds of the invention in combination with water, isopropanol, ethanol, methanol, dimethyl sulfoxide, ethyl acetate, acetic acid, or ethanolamine.

A “pharmaceutically acceptable salt” is intended to mean a salt that retains the biological effectiveness of the free acids and bases of the specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophaosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1, 4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

The present invention further provides synthetic methods that are comprised of one of the synthetic steps set forth in the present disclosure. A synthetic method is comprised of a synthetic step when the synthetic step is at least part of the final synthetic method. In such a fashion, the synthetic method can be only the synthetic step or have additional synthetic steps that may be associated with it. Such a synthetic method can have a few additional synthetic steps or can have numerous additional synthetic steps.

If the antipicornaviral agent of formula I formed from the process of the present invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as apsartic acid or glutamic acid; aromatic acid, such as benzoic acid or cinnamic acid; sulfonic acid, such as p-toluenesulfonic acid or ethanesulfonic acid; or the like.

If the antipicornaviral agent of formula I formed from the process of the present invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of compounds, salts, or solvates that are solids, it is understood by those skilled in the art that the compounds of formula I and the intermediates used in the process of the present invention, salts, and solvates thereof, may exist in different crystal forms, all of which are intended to be within the scope of the present invention and specified formulas.

The antipicornaviral agents of formula I, and the intermediates used in the process of the present invention, may exist as single stereoisomers, racemates, and/or mixtures of enantiometers and/or diastereomers. All such single stereoisomers, racemates, and mixtures thereof are intended to be within the broad scope of the present invention. Preferably, however, the intermediate compounds used in the process of the present invention are used in optically pure form.

As generally understood by those skilled in the art, an optically pure compound is one that is enantiomerically pure. As used herein, the term “optically pure” is intended to mean a compound comprising at least a sufficient amount of a single enantiomer to yield a compound having the desired pharmacological activity. Preferably, “optically pure” is intended to mean a compound that comprises at least 90% of a single isomer (80% enantiomeric excess), more preferably at least 95% (90% e.e.), even more preferably at least 97.5% (95% e.e.), and most preferably at least 99% (98% e.e.) Preferably, the antipicornaviral agents of formula I formed from the process of the present invention are optically pure.

The present invention relates to a process of preparing antipicornaviral agents of formula I:

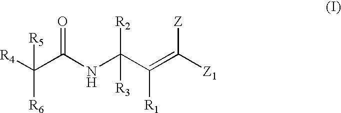

wherein R₁ is H, F, an alkyl group, OH, SH, or an O-alkyl group; R₂ and R₃ are each independently H;

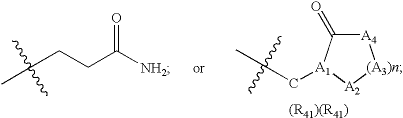

where n is an integer from 0 to 5, A₁ is CH or N, A₂ and each A₃ are independently selected from C(R₄₁)(R₄₁), N(R₄₁), S, S(O), S(O)₂, and O, and A4 is NH or NR₄₁, where each R₄₁ is independently H or lower alkyl, provided that no more than two heteroatoms occur consecutively in the above-depicted ring formed by A₁, A₂, (A₃)ₙ, A₄ and C═O, and at least one of R₂ and R₃ is

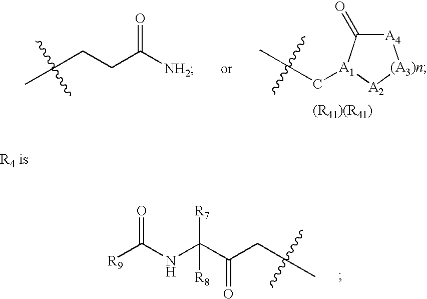

R₅ and R₆ are each independently H, F, an alkyl group, a cycloalkyl group, a heterocycloalkyl group, an aryl group, or a heteroaryl group;

R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈, where R₁₇, R₁₈, and R₁₉ are each independently H, an alkyl group, a cycloalkyl group, a heterocycloalkyl group, an aryl group, a heteroaryl group, or an acyl group, provided that at least one of R₇ and R₈ is an alkyl group, an aryl group, a heteroaryl group, —OR₁₇, —SR₁₇, —NR₁₇R₁₈, —NR₁₉NR₁₇R₁₈, or —NR₁₇OR₁₈;

R₉ is a five-membered heterocycle having from one to three heteroatoms selected from O, N, and S; and

Z and Z₁ are each independently H, F, an alkyl group, a cycloalkyl group, a heterocycloalkyl group, an aryl group, a heteroaryl group, —C(O)R₂₁, —CO₂R₂₁, CN, —C(O)NR₂₁R₂₂, —C(O)NR₂₁OR₂₂, —C(S)R₂₁, —C(S)NR₂₁R₂₂, —NO₂, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), —PO(NR₂₁R₂₂)(NR₂₃R₂₄), —C(O)NR₂₁NR₂₂R₂₃, or —C(S)NR₂₁NR₂₂R₂₃, where R₂₁, R₂₂, R₂₃, and R₂₄ are each independently H, an alkyl group, a cycloalkyl group, a heterocycloalkyl group, an aryl group, a heteroaryl group, an acyl group, or a thioacyl group, or where any of two of R₂₁, R₂₂, R₂₃, and R₂₄, together with the atom(s) to which they are bonded, form a heterocycloalkyl group, provided that Z and Z₁ are not both H;

or Z₁ and R₁, together with the atoms to which they are bonded, form a cycloalkyl or heterocycloalkyl group, where Z₁ and R₁ are as defined above except for moieties that cannot form the cycloalkyl or heterocycloalkyl group;

or Z and Z₁, together with the atoms to which they are bonded, form a cycloalkyl or heterocycloalkyl group, where Z and Z₁ are as defined above except for moieties that cannot form the cycloalkyl or heterocycloalkyl group.

The present invention discloses that a compound of formula I may be prepared by subjecting a compound of formula II and a compound of formula III to an amide-forming reaction:

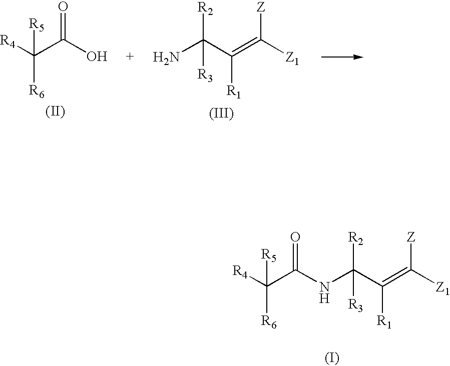

The amide-forming reaction may be achieved by any suitable method, reagents and reaction conditions. Preferably, any one of the methods disclosed in the '354 application is utilized. For example, a compound of formula II may be reacted with a compound of formula III in the presence of HATU, DIPEA, CH₃CN and H₂O to yield the desired compound of formula I. Any suitable purification method may be used to further purify the compound of formula I.

More preferably, the compound of formula I is prepared by an amide-forming reaction comprising the steps of:

(a) reacting the compound of formula II with a compound of formula IIIA in the presence of N-methylmorpholine to form a reaction mixture; and

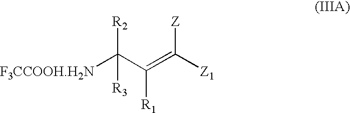

(b) adding a compound of formula Lv-X to the reaction mixture to form a compound of formula I, wherein X is any suitable halide.

Preferably, the method for preparing the compound of formula I utilizing the more preferable amide-forming reaction utilizes some or all of the reagents and reaction conditions disclosed below. Thus, preferably, the compound of formula II and the compound of formula IIIA in DMF are combined in any suitable container. The suitable container is preferably a single neck flask which is then covered with any suitable septum and covered with a temperature probe. Nitrogen gas is used to purge out the suitable container before N-methylmorpholine is added to the reaction mixture. More preferably, the N-methylmorpholine is added via a syringe in one single portion and the reaction mixture cooled to about between −5° C. and 5° C. More preferably, the reaction mixture is cooled to about 0° C. A solution of the compound of formula Lv-X is then added to the reaction mixture. More preferably, the solution of the compound of formula Lv-X is a solution of the compound of formula Lv-X in DMF. Even more preferably, the compound of formula Lv-X is CDMT. The solution of the compound of formula Lv-X is added to the reaction mixture by any suitable method so as to maintain the reaction mixture at a constant temperature. For example, the solution of the compound of formula Lv-X may be added to the reaction mixture dropwise utilizing a syringe. Upon completion of the addition of the solution of the compound of formula Lv-X, the reaction mixture is allowed to warm to about room temperature. The progress of the reaction may be followed by monitoring the disappearance of the compound of formula II by thin layer chromatography (hereinafter “TLC”). When the reaction is at least substantially complete, the compound of formula I may be precipitated out of solution to form a slurry by slowly adding water to the reaction mixture. The compound of formula I may then be removed from the slurry by any suitable means known to one of ordinary skill in the art. For example, the compound of formula I may be removed from the slurry by filtration. Any suitable purification method known to one of ordinary skill in the art may be used to purify the compound of formula I. More preferably, the compound of formula I is purified by recrystalization.

Any method known to one of ordinary skill in the art may be used to prepare the compound of formula IIIA. However, the present invention also discloses a novel method for preparing the compound of formula IIIA comprising the step of reacting a compound of formula IIIB with TFA:

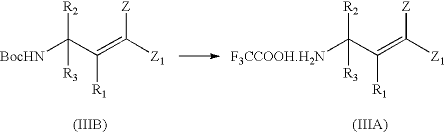

Preferably, the method for preparing the compound of formula IIIA from the compound of formula IIIB utilizes some or all of the reagents and reaction conditions disclosed below. Thus, preferably, the compound of formula IIIB and DCM are placed into a suitable container and covered with a septum. The container is then purged with nitrogen followed by the addition of TFA. More preferably, the TFA is added via syringe while stirring. The progress of the reaction may be followed by TLC. Once the starting material has substantially disappeared the solvent and excess TFA are removed by any suitable means. For example, the solvent and excess TFA may be removed by vacuum distillation. Preferably, the compound of formula IIIA is used immediately in the process of the present invention to prepare the compound of formula I.

The present invention also relates to a process for the preparation of the compounds of formula IIA:

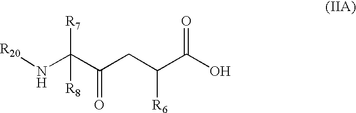

One of ordinary skill in the art will recognize that the compounds of formula IIA fall within the genus as defined by formula II. Accordingly, the compounds of formula IIA are also useful intermediates for preparing the antipicornaviral agents of formula I.

The present invention discloses a process for preparing the compounds of formula IIA, comprising the steps of:

(a) the conversion of a compound of formula XIII to a β-ketoester of formula XIV by reacting it with a 1,1′-carbonyldiimidazole and a lithium enolate of t-butyl acetate;

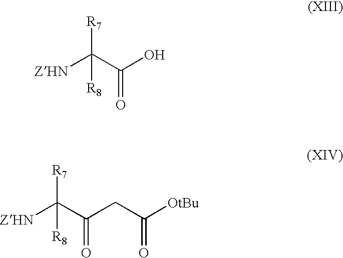

(b) the conversion of the compound of formula XIV to an enolate of formula XV by reacting it with a compound of formula XVI under suitable reaction conditions;

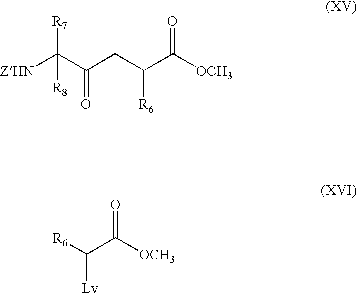

(c) the hydrogenolysis of formula XV to yield a compound of formula XVII;

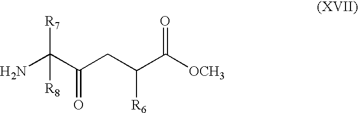

(d) the acylation of the compound of formula XVII by reacting a compound of formula R₂₀-X under suitable conditions to yield a compound of formula XVIII, wherein X is a halide; and

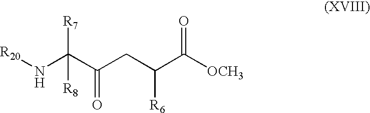

(e) the enzymatic hydrolysis of the compound of formula XVIII to yield the compound of formula IIA.

Preferably, the method for converting the compound of formula XIII to that of formula XIV utilizes some or all of the reagents and reaction conditions disclosed below. Thus, preferably, the compound of formula XIII is stirred with CDI in THF under a nitrogen stream for at least about 1 hour at room temperature to yield an acyl imidazole intermediate. Then, in a separate container, lithium bistrimethylsilylamide solution (LHMDS) is charged in THF under nitrogen, prior to cooling to −70° C. t-Butyl acetate is added slowly to the LHMDS solution keeping the temperature below about −60° C. to form a reaction mixture. The acyl imidazole intermediate prepared as disclosed above, is slowly added to the reaction mixture, comprising the lithium enolate of t-butyl acetate, under nitrogen keeping the internal temperature at or below about −60° C. Once this addition is completed, the reaction mixture is stirred at −60° C. for at least an additional 1 hour. The reaction mixture is then charged with 1M HCl to quench the reaction. The HCl is added slowly, with rigorous stirring, keeping the internal temperature of the reaction mixture below about −50° C. Higher temperatures during quenching causes racemization. Concentrated HCl is added to adjust the pH to about between 6-7.5. Any solids that precipitate out are filtered off. Because warmer temperatures dissolve impurities the filtration is more preferably carried out cold and rapidly over celite. The solids are then washed with MTBE. The filtrate is diluted with MTBE and HCl and agitated for at least about 15 minutes. The pH should be checked to ensure that a pH is about between 1-2. After the organic layer is separated, it may be checked for chiral purity by chiral HPLC. If chirally pure products are desired, the chiral purity should be about 98% at this stage. The organic layer is washed, preferably with 1M HCl and agitated for about 15 minutes prior to the layers being separated. The organics are washed, preferably with saturated sodium bicarbonate solution and agitated for at least about 15 minutes, before the layers are separated. The organic layer is then washed, preferably with brine. The phases are separated before the organic layer is dried, preferably over anhydrous magnesium sulfate. It is then filtered and striped under vacuum to remove solvents and unreacted t-butyl acetate. A high-vacuum is maintained for at least about 20 hours to ensure the removal of t-butyl acetate and siloxanes. At this stage, the product may be analyzed for purity. Should the product be significantly less than about 90% pure, the product can be chromatographed over silica using 20% ethyl acetate/hexanes. Under these preferable conditions, yields of between 60 and 88% of compound XIV are attainable.

The conversion of the compound of formula XIV to that of compound of formula XV by reacting it with the compound of formula XVI may be carried using any suitable method, reagents and reaction conditions. An example of this general method is disclosed in R. V. Hoffman and J. Tao, Tetrahedron, Vol. 53, No. 21, pp. 7119-7126, 1997, which is herein incorporated by reference in its entirety. Preferably, the method and all or some of the reagents and reaction conditions disclosed below are used. Thus, preferably, the compound of formula XIV is first reacted with an alkali metal hydride before reacting it with the compound of formula XVI. More preferably the alkali metal hydride is sodium hydride. The reaction with the alkali metal hydride is conducted at between about 0° C. and 3° C. The reagents are kept at between about 0° C. and 5° C. during the addition of compound of formula XVI to the reaction mixture, before the reaction mixture is slowly being warmed to ambient temperature over at least about 2 hours.

Any suitable hydrogenolysis method may be used to convert compound XV to compound XVII. Preferably, palladium hydrogenolysis under pressure is used.

Any suitable reaction conditions may be used in the acylation of compound XVII. Preferably, the method and some or all of the reagents and reaction conditions disclosed hereinafter are utilized. Thus, preferably, the crude compound of formula XV is dissolved in methylene chloride and cooled to about 0° C. (internal temperature) by any suitable means, for example, using an ice/salt bath under a blanket of argon. The solution is charged with the compound of formula R₂₀-X as a liquid. More preferably, R₂₀-X is R₂₀-Cl. Diisopropylethyl amine is then added slowly. The reaction is allowed to slowly warm to room temperature. The reaction may be monitored by TLC and finally by HPLC. Generally this reaction should be complete within about 1 hour. The reaction is quenched with HCl, before the aqueous layer is removed and the organics are reextracted with HCl. The aqueous phase is then removed before the organics are extracted with saturated bicarbonate. The organics are then dried, preferably, over sodium sulfate. The product is then filtered and concentrated under vacuum.

Any suitable enzymatic hydrolysis method may be used to convert compound XIII to compound IIA. However, the present invention discloses that the use of enzymatic hydrolysis is important as opposed to hydrolysis under standard conditions, because it produces compound IIA with less than 5% epimer at the carbon linking the R₇ and R₈ groups. Any suitable apparatus may be used in the enzymatic hydrolysis step. Preferably a continuous membrane reactor is used. More preferably, the continuous membrane reactor of the present invention is used as disclosed hereinafter.

Preferably, porcine pancrease lipase is used as the enzyme to hydrolyze compound XVIII. More preferably, the enzymatic hydrolysis is conducted at a pH of about 7.2 at a temperature of between about 37-40° C.

Another aspect of the present invention is the preparation of the compounds of formula IIA by a process comprising the steps of:

(a) the conversion of a compound of formula XIX to the β-ketoester of formula XX by reacting it with 1,1′-carbonyldiimidazole followed by treatment with lithium enolate of t-butyl acetate;

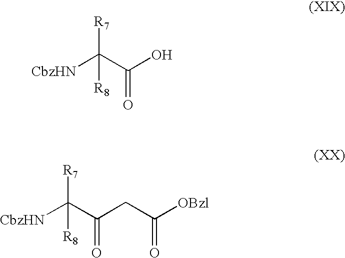

(b) the conversion of the compound of formula XX to a compound of formula XXI by reacting it with a compound of formula XXII under suitable reaction conditions;

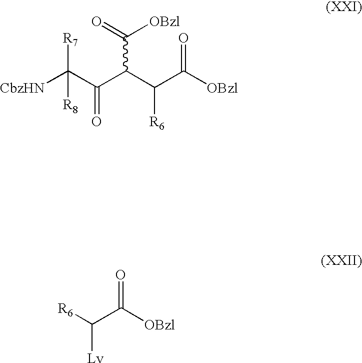

(c) the hydrogenation of the compound of formula XXII to yield a compound of formula XXIII; and

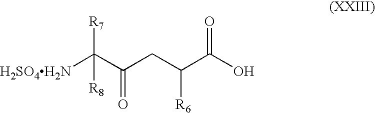

(d) the acylation of the compound of formula XXIII by reacting it with R₂₀-X under suitable conditions to yield the compound of formula IIA, wherein X is any suitable halide.

Preferably, the method for converting the compound of formula XIX to the compound of formula XX utilizes some or all of the reagents and reaction conditions disclosed below. Thus, preferably the compound of formula XIX is dissolved in THF before the 1,1′-carbonyldiimidazole is added to the solution at room temperature. The resulting mixture is stirred for about 1 hour at room temperature to yield a solution of an acyl imidazole intermediate.

In a separate container, σ-benzyl acetate is slowly added to a solution of LiHMDS in THF to form a mixture. The reaction is exothermic, therefore the temperature is preferably maintained below −70° C. After stirring the mixture for about 30 minutes the acyl imidazole solution is slowly added to it to form a reaction mixture. The reaction is exothermic, thus, the temperature of the reaction mixture is preferably maintained under about −68° C. Any suitable means for cooling the reaction mixture may be used. For example, the cooling means may be a dry ice bath. After stirring for at least about 55 minutes the reaction mixture may be removed from the cooling means. An acid is then added to the reaction mixture to quench the reaction. More preferably, the acid is 1M HCl, the acid is added slowly, and the temperature of the reaction mixture is maintained at under about 25° C. during the addition of the acid. The organic layer of the quenched reaction mixture is then separated and washed. More preferably, the organic layer is washed with saturated sodium bicarbonate and brine. The organic layer is then dried and concentrated to yield the compound of formula XX. More preferably, magnesium sulfate is used as the drying agent. To prevent decomposition of the compound of formula XX, the compound is more preferably stored in a refrigerator.

Preferably, the method for converting the compound of formula XX to the compound of formula XXI utilizes some or all of the reagents and reaction conditions disclosed below. Thus, preferably the compound of formula XX is slowly added to a solution of NaH in THF. More preferably, the solution of NaH in THF is maintained at about −10° C. whilst the compound of formula XX is added to it. Once the compound of formula XX has been added to the solution, the reaction mixture is allowed to warm for about 20 minutes. A solution of the compound of formula XXII in methylene chloride is then added to the reaction mixture. The progress of the reaction may be monitored by observing the disappearance of the starting materials using any suitable method. For example, HPLC may be used to monitor the progress of the reaction. The reaction mixture is then stirred for about 48 hours before MTBE is added to it. A suitable acid is then added to the reaction mixture before the aqueous layer is separated and extracted using MTBE. More preferably, the acid is 1M HCl. The organic layers are then combined, dried, filtered and concentrate to yield the compound of formula XXI. More preferably, the combined organic layer is dried in magnesium sulfate and filtered through a short pad of silica gel.

Preferably, the method for converting the compound of formula XXI to the compound of formula XXIII utilizes some or all of the reagents and reaction conditions disclosed below. Thus, preferably the compound of formula XXI is dissolved in a degassed mixture of THF and concentrated acid. More preferably, the concentrated acid is sulfuric acid. 10% Pd-C is added to the reaction mixture before the mixture is stirred in a Parr shaker under a pressure at about 50 psi for about 5 hours. The mixture is then dissolved in methanol, filtered through celite to yield the compound of formula XIII.

Preferably, the method for converting the compound of formula XXIII to the compound of formula IIA utilizes some or all of the reagents and reaction conditions disclosed below. Thus, preferably the compound of formula XXIII is dissolved in dioxane, followed by the addition of diisopropylethylamine to form a suspension at 0° C. A solution of the compound of formula R₂₀-X in dioxane at a similar temperature to that of the suspension is added to the suspension to form a reaction mixture. More preferably, R₂₀-X is R₂₀-Cl. The reaction mixture is then stirred for at least about 1 hour. Then, methylene chloride is added to the reaction mixture before the reaction mixture is washed with 1M HCl then saturated sodium bicarbonate, dried with magnesium sulfate and filtered through a short pad of silica gel to yield the compound of formula IIA.

The compound of formula IIA may then be purified by any means known to one of ordinary skill in the art. For example, the compound may be purified by recrystallization and/or chromatography.

The present invention also relates to an improved process for the preparation of the compound of formula XXII. As disclosed above, the compound of formula XXII is an important starting material for use in the process for preparing the compound of formula IIA. The process of preparing the compound of formula XXII comprises:

(a) reacting a compound of formula XXIV with triethylamine and benzyl bromide to give a compound of formula XXV; and

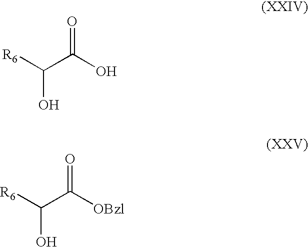

(b) converting the compound of formula XXV to the compound of formula XXII.

Preferably, the method for converting the compound of formula XXIV to the compound of formula XXV utilizes some or all of the reagents and reaction conditions disclosed below. Thus, preferably the compound of formula XXIV is dissolved in acetone, followed by the slow addition of triethylamine at a temperature less than about 30° C. to form a reaction mixture. Benzyl bromide is then added to the reaction mixture which is then stirred for at least about 65 hours. MTBE is then added to the reaction mixture and stirred for about 5 minutes. The reaction mixture is then filtered through a short pad of silica gel to remove most of a triethylamine salt which precipitates out of the reaction mixture. Then the silica gel is washed with MTBE before the filtrates are combined. The combined filtrate is then washed. More preferably the filtrate is washed with 1M HCl, saturated sodium bicarbonate and brine. Then the filtrate is dried in magnesium sulfate, filtered through a short pad of silica gel and concentrated to give the compound of formula XXV. The compound of formula XXV may be recrystalized to give a crystalline product.

Preferably, the method for converting the compound of formula XXV to the compound of formula XXII utilizes some or all of the reagents and reaction conditions disclosed below. Thus, preferably the compound of formula XXV is dissolved in methylene chloride and cooled to about −10° C. Although any suitable leaving group may be substituted with the hydroxy group of the compound of formula XXV to yield a compound of formula XXII, preferably the leaving group is —OTf. Accordingly, more preferably, Tf₂O is added to the solution of the compound of formula XXV in methylene chloride, followed by the slow addition of 2,6-lutidine. Because the reaction is exothermic the temperature of the reaction mixture is preferably maintained at a temperature of under about −8° C. Once the 2,6-lutidine has been added to the reaction mixture, the reaction mixture is stirred and allowed to warm for about 1 hour. The reaction mixture is then concentrated under house vacuum. The crude product, normally in the form of an oil, is then dissolved in hexanes and stirred on dry ice to precipitate out a lutidinium salt. The precipitate is then removed by filtration through a thin layer of silica gel. The filtrate is then concentrated to yield the compound of formula XXII wherein the leaving group Lv is —OTf.

The present invention also relates to novel compounds falling within the scope the compounds of formulae IIA; XVIII; XVII; XV; IIIB and IIIA respectively. These particular compounds set forth below are particularly useful as intermediates in the process of the present invention to synthesize particularly useful antipicornaviral compounds of the general formula I, including AG7088:

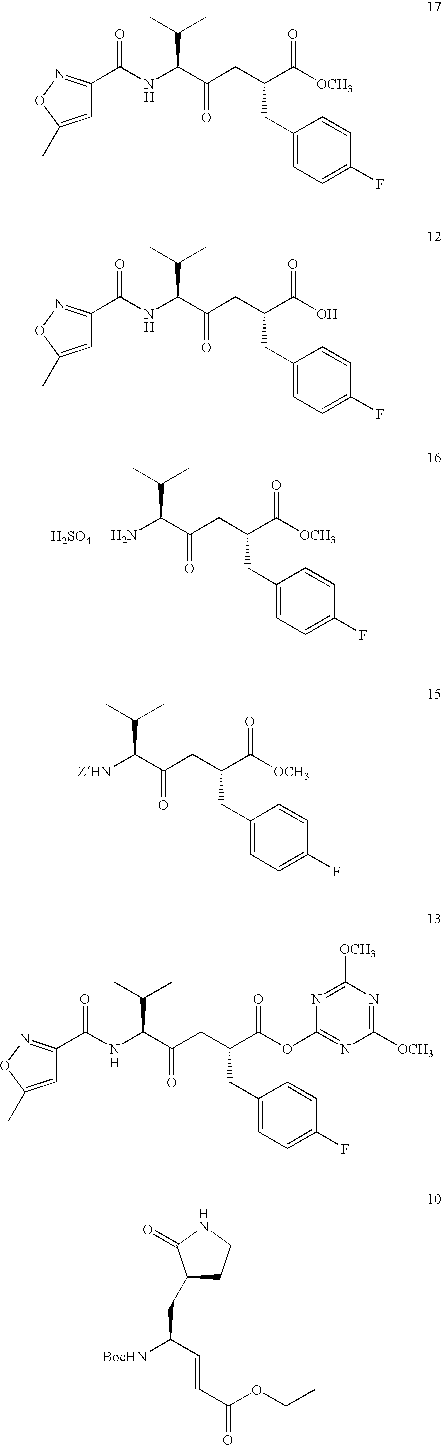

Another aspect of the present invention relates to improved processes for preparing compounds falling within the scope of formulae XXIV and XVI, key reagents in the process of the present invention for preparing compounds of formula IIA.

The first of these is a process for the preparation of compounds of formulae VII falling within the scope of the compounds of formula XXIV and optionally the conversion of the compound of formula VII to the compound of formula XVIA, the scope of which overlaps with the compounds of formula XVI:

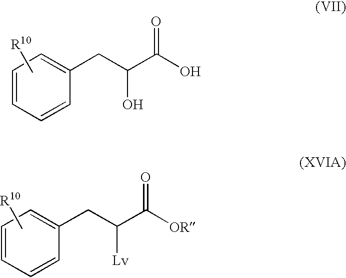

wherein R₁₀ is a halogen or an alkyl group;

comprising the steps of:

Step A: converting a compound of formula VI to a compound of formula V comprising the substeps of:

(a) reacting a R₁₀ substituted benzaldehyde of formula VI:

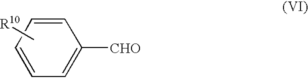

with hydantoin in an aqueous medium in the presence of a catalyst at reflux temperature to form a reaction mixture;

(b) treating the reaction mixture with an excess of an alkali metal hydroxide at reflux temperature to form a alkali metal hydroxide-treated solution;

(c) adding an alkali metal halide to the alkali metal hydroxide-treated solution to give a solution;

(d) acidifying the solution with a concentrated acid to give a precipitate of formula V;

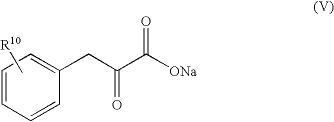

and

(e) optionally washing the precipitate of formula V with a washing agent;

Step B: the enzymatic reduction of the compound of formula V to a compound of formula VII;

Optional step C: an esterification of the compound of formula VII to a compound of formula XII by reacting the compound of formula VII with a compound of formula R″—OH, wherein R″ is an alkyl or aryl; and

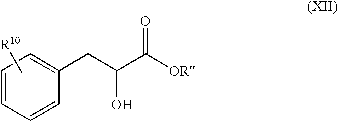

Optional step D: the conversion of the compound of formula XII to the compound of formula XVIA.

Thus, the present invention discloses that the reaction of the R₁₀ substituted benzaldehyde with the hydantoin in an aqueous medium in the presence of a catalytic quantity of a primary or secondary amine under reflux for at least about 4 hours, depending upon the amine used, will yield R₁₀ substituted 5-benzylidene hydantoin. The preferred amines have boiling points above that of the aqueous medium used. A particularly preferable amine is 1-amino-2-propanol. When 1-amino-2-propanol is used as a catalyst, water is used as the aqueous solution and the molar ratio of the R₁₀ substituted benzaldehyde to hydantoin to the catalyst is 1:1:0.1, the reaction is completed in about 4 hours.

According to the present invention the R₁₀ substituted 5-benzylidene hydantoin can be hydrolyzed by an excess amount of an alkali metal hydroxide. Preferably, the alkali metal hydroxide used is sodium hydroxide. When 1-amino-2-propanol is used as a catalyst, the molar ratios of sodium hydroxide to hydantoin are individually 5:1, and the reaction is carried out under reflux, the reaction is completed in about 50 minutes.

The present invention also discloses that the addition of an alkali metal halide to the alkali metal hydroxide-treated solution increases the precipitation of monohydrated alkali metal R₁₀ substituted phenylpyruvate upon acidification. Preferably, the alkali metal halide is sodium chloride. When sodium chloride is used, almost all the sodium phenylpyruvate precipitates out as monohydrated sodium phenylpyruvate at a pH of about 8.5.

Preferably, the collected monohydrated alkali-metal phenylpyruvate precipitate is washed to remove excess impurities and to facilitate the drying process should that be desired. Any suitable washing agent known in the art may be selected. Preferably a primary alcohol is selected as the washing agent. More preferably, the washing agent is methanol because the monohydrated alkali-metal phenylpyruvate precipitate is sparingly soluble therein.

Any suitable enzyme known in the art may be used in Step B to catalyze the reduction reaction of the compound of formula V. Preferably, the reduction reaction is catalyzed by formate dehydrogenase and lactate dehydrogenase.

Any suitable enzymatic reduction method known in the art may be used. Preferably, either the membrane-enclosed enzymatic catalysis method (“the MEEC method”) or the coimmobilization method is used. These general methods are known in the art. For example, see Bednarski et al., J. Am. Chem. Soc. 1987, 109, 1283-1285, for a general discussion regarding membrane-enclosed enzymatic catalysis. See also Pollak et al., J. Am. Chem. Soc. 1980, 102, 6324-6336, for a general discussion of the coimmobilization method. These references are incorporated herein by reference in their entirety. However, when the enzymatic reduction reaction of step B involves more than a small scale preparation, preferably a continuous membrane reactor is employed. More preferably, the continuous membrane reactor of the present invention is used. When the continuous membrane reactor of the present invention is used, preferably all or some of the following reagents and conditions are used: 1% NAD, 4 equivalents of ammonium formate, a pH of 7.3-7.4 for the effluents and a pH of 6.2-6.3 for the substrates, FDH/LDH=20/200 (U/mL) and 1 mM mercaptoethanol are used.

If the coimmobilization method is used, it is preferably carried out in four steps. The first step is the preparation of N-acryloxysuccinimide. The second step is the preparation of the copolymer for use in the coimmobilization method. Preferably, the copolymer is PAN 500 which may be prepared by a radical coploymerization. One of ordinary skill in the art will recognize that PAN 500 is a water soluble copolymer of acrylamide and N-acryloxysuccinimide which releases 500 (±25) μmol of N-hydroxysuccinimide per gram of dry polymer on treatment with excess aqueous ethylamine solution. The third step is the coimmobilization of the enzymes. Preferably, as disclosed above, the enzymes are formate dehydrogenase and lactate dehydrogenase. The fourth step is the enzymatic reduction of the reduction of the compound of formula V to give the compound of formula VII.

The compound of formula VII may be isolated at this stage of the process and used in the process disclosed above for preparing the compound of formula IIA. Any suitable method may be used to isolate and purify the compound of formula VII. Optionally, the compound of formula VII may be used to prepare the compound of formula XVIA as disclosed below.

The present invention also discloses that if enantiomeric forms of a compound of formula VII is sought, the use of D-lactate dehydrogenase in Step B described above will yield an enantiomer of formula VIIA:

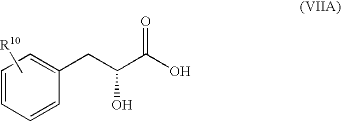

Similarly, the use of L-lactate dehydrogenase in Step B described above will yield an enantiomer of formula VIIB:

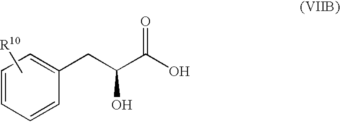

The esterification reaction of optional step C may be performed with any suitable reagents and conditions. Preferably, the esterification is performed at about room temperature in the presence of hydrochloric acid and dioxane.

Similarly, the enantiomers VIIA and VIIB may be converted to enantiomers XIIA and XIIB respectively by the same esterification process:

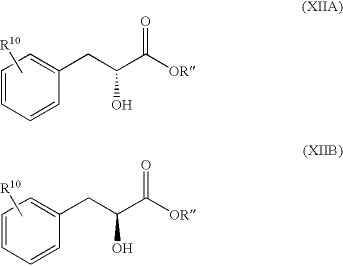

Any suitable method may be used to convert the compound of formula XII to the compound of formula XVIA in optional step D of the present process. For example suitable methods are disclosed in Efffenberger et al., J. Liebigs. Ann. Chem. 1996, 314, and “Peptidomimetics Protocols”, Hoffman et al., Human Press, NJ, U.S.A; 1999, pp 103-124. These references are herein incorporated by reference.

Utilizing this same optional step D, enantiomers XIIA and XIIB may be converted to enantiomers XVIB and XVIC respectively:

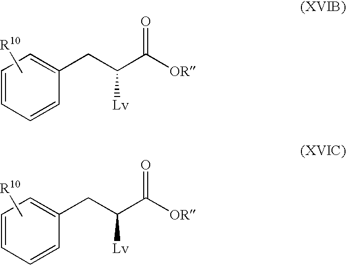

The second of the processes for preparing compounds of formulae VII and XVIA comprises the steps of:

Step A′ converting serine to the compound of formula VII comprising the substeps of:

(a) converting serine to potassium glycidate by a standard process;

(b) optionally converting the potassium glycidate to a glycidic acid; and

(c) carrying out a regioselective epoxide ring-opening reaction with a compound of formula R₁₀-phenyl-Q;

wherein Q is an activated bromide, a sulfate, or a primary iodide;

Optional step B′: an esterification of the compound of formula VII to a compound of formula XII by reacting the compound of formula VII with a compound of formula R″—OH, wherein R″ is an alkyl or aryl; and

Optional step C′: the conversion of the compound of formula XII to the compound of formula XVIA.

Accordingly, substep (a) of Step A′ of this process requires the conversion of serine to potassium glycidate by a standard process. Any such standard process known in the art may be used. For example, Larchevêque et al., Tetrahedron Lett. 1987, 28, 1993-1996, discloses the preparation of potassium glycidate from serine. This reference is incorporated herein by reference in its entirety.

Preferably, serine is reacted with nitric acid at a suitable temperature to yield 2-bromo 3-hydroxy propanoic acid. More preferably, the nitrous acid comprises a mixture of sodium nitrate and hydrogen bromide, and the reaction is carried out at between about −10° C. and room temperature in the presence of an alkali metal halide. Any suitable alkali metal halide known in the art may be used. However, preferably, the alkali metal halide is potassium bromide or sodium bromide.

The 2-bromo 3-hydroxy propanoic acid is then converted to potassium glycidate by reacting it with potassium hydroxide. Preferably the reaction is run at between about −40° C. and room temperature.

When the preferred conditions and reagents are used in accordance with the present invention, a 65-70% yield of potassium glycidate from serine is attainable.

The present invention also discloses that the use of enantiomeric L-serine or D-serine as the starting material in the process described above will yield D-potassium glycidate and L-potassium glycidate respectively.

The potassium glycidate from the process disclosed above may be converted directly into the compound of formula VII. Reacting potassium glycidate with a compound of formula R₁₀-phenyl-Q will cause a regioselective epoxide ring-opening reaction. Preferably, Q is an —MgBr group and the regioselective ring-opening reaction is performed at between about −10° C. and room temperature in the presence of copper iodide.

Instead of converting the potassium directly into a compound of formula VII, the potassium glycidate may first be converted to glycidic acid before being converted to a compound of formula VII by the epoxide ring-opening method described above. The potassium glycidate may be converted to glycidic acid by any method known to one of ordinary skill in the art. Preferably, the glycidic acid is prepared by reacting the potassium glycidate with concentrated nitric acid.

If enantiomeric potassium glycidate is used in the methods described above, the corresponding enantiomer of the compound of formula VII will be synthesized. For example, if D-potassium glycidate is used, a compound of formula VIIA will be formed. Similarly, if L-potassium glycidate is used, a compound of formula VIIB will be formed.

At this stage, the compound of formula VII may be isolated for use in the process disclosed above for preparing the compound of formula IIA. Alternatively, the compound of formula VII may be used in the process disclosed below to prepare the compound of formula XVIA.

Optional steps B′ and C′ correspond to optional steps C and D of the first disclosed process for synthesizing the compound of formula XVIA from a compound of formula VI respectively. Thus, the preferred methods, reagents and reaction conditions disclosed above for optional steps C and D are also preferably used in optional steps B′ and C′.

The third of the processes for preparing a compound falling within the scope of formula XVIA, specifically a compound of formula XVIB, comprises the steps of:

Step A″: the preparation of a compound of formula XIIA from a compound of formula IX comprising the substeps of:

(a) an asymmetric dihydroxylation of a compound of formula IX to form a compound of formula XA:

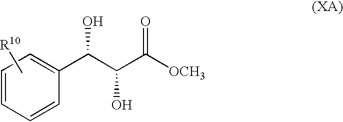

(b) reacting the compound of formula IX with 1,1′-carbonyldiimidazole in the presence of toluene to form a compound of formula XI; and

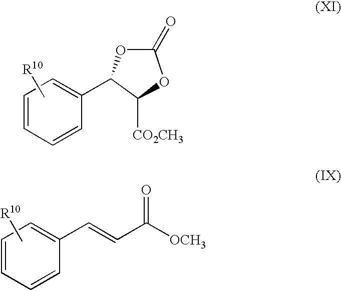

(c) a palladium-mediated reduction of the compound of formula XI; and

Step B″ the conversion of the compound formula XIIA to the compound of formula XVIB.

Preferably, the asymmetric dihydroxylation is a Sharpless asymmetric dihydroxylation performed at about room temperature. Asymmetric dihydroxylation, including Sharpless asymmetric dihydroxylation is discussed generally in Kolb et al., Chem. Rev. 1994, 94, 2483-2547. This reference is herein incorporated by reference in its entirety.

Preferably, the reaction of the compound of formula IX with CDI in the presence of toluene is performed at about 80° C.

Preferably, the palladium-mediated reduction step, Step A″ (c), is performed by reacting the compound of formula XI with a mixture of hydrogen, palladium and carbon in the presence of formic acid at about room temperature.

Step B″ corresponds to optional step D of the herein first disclosed process for synthesizing the compounds of formula XVIA from a compound of formula VI. Thus, the same method, reagents, and reaction conditions disclosed for use in optional step D are preferably also used in Step B″.

The present invention also relates to the compounds of formula IVA, falling within the scope of the genus defined by formula IV as recited above. Accordingly, the compounds of formula IVA will also be useful intermediates in the processes of the present invention for the preparation of compounds of formula I.

Thus, the present invention relates to a compound of formula IVA:

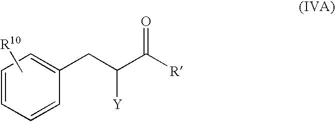

Y is OH, OSO₂CF₃, OSO₂CH₃, OSO₂(p-tolyl), halide or any other leaving group; and R′ is H, alkyl or aryl group.

Preferably, R₁₀ is a 4-fluoro group, Y is OH or OTf, and R′ is OH or Me.

As discussed above, the present invention also relates to a continuous membrane reactor that can be used in the processes of the present invention. In particular, the continuous membrane reactor of the present invention is suitable for use in any reaction in which a catalyst of a relatively large molecular size is employed, such, as enzymes and anchored catalysts. Examples of such catalysts are disclosed in Rissom et al., Tetrahyedron: Asymmetry, 1999, 10, 923-928; Schmidt et al., J. Biotechnology, 1992, 24, 315-327; and Lin et al., Biosci. Biotech. Biochem., 1997, 61, 2029-2033. The aforementioned references are herein disclosed by reference. More particularly, the continuous membrane reactor of the present invention is of use in those catalytic reactions in which there is a desire to recycle the catalyst. For example, the reactor of the present invention is useful for use in enzymatic reduction reactions utilizing either chemical or bio-catalysts.

The continuous membrane reactor of the present invention having a reactor volume comprises a tangential flow filter unit, a reactor loop to circulate the reagents through the tangential flow filter, and a substrate feed pump for feeding the substrate into the reactor loop, wherein the reactor loop comprises:

(a) a tube; and

(b) a circulation pump.

The tangential flow unit comprises a tangential flow membrane filter and a unit for housing the filter. Any suitable tangential flow unit may be used. A suitable tangential flow unit is one which allows the desired product, or permeate, to pass through it but retains the larger molecules of the catalyst in the reactor. A preferred example of the tangential flow unit is the Pellicon 2 Module commercially available from Millipore Corporation. The Pellicon 2 Module employs a cassette style tangential flow filtration device which allows for easy scale-up of the reaction. Specifically, either a single Pellican 2 cassette may be employed or a series of cassettes can be used in combination to allow for a larger scale reaction to be run. Thus, the utilization of a tangential flow cartridge system allows for the processing of fluid volumes from less than a liter up to thousands of liters.

The reactor loop, in which the majority of the catalyzed reaction occurs, has an internal volume. The internal volume is defined by the volume of reagents and catalyst the reactor loop can hold. The reactor volume is defined by the volume of reagents and the catalyst the reactor loop and the tangential flow unit in combination can hold. The reactor loop of the reactor of the present invention has an internal volume of at least about 50% of the reactor volume. Preferably, the reactor loop has an internal volume of at least about 60% of the reactor volume. More preferably, the reactor loop has an internal volume of at least about 70% of the reactor volume. Even more preferably, the reactor loop has an internal volume of at least about 80% of the reactor volume. In a more preferred embodiment of the present invention, the reactor loop has an internal volume of at least about 90% of the reactor volume. In a more preferred embodiment of the present invention, the reactor loop has an internal volume of at least about 95% of the reactor volume.

The reactor loop comprises a tube of any suitable size and made from any suitable material. Preferably, the reactor loop comprises tubing which is flexible. Flexible tubing allows for the tubing to be cut to any desired length as a means for easily varying the reactor volume. Examples of suitable tubing materials include polyethylene, polypropylene, polyurethane, polyvinyl, vinyl, nylon, butylene-polymer, silicone PTFE, ETFE, PFA, Viton®, stainless steel, glass, PVDF, Teflon®, an alkyl polymer, and a perfluoro material. Viton® is commercially available from Dupont Dow Elastomers LLC and comprises a 67% fluorinated thermal set rubber. Teflon® is commercially available from EI Dupont deNemours & Co. and comprises tetrafluoro ethylene. When the continuous membrane reactor of the present invention is used in the process of the present invention to carry out an enzymatic reduction, it has been found that PVC, Tygon® and any chlorinated polymer damage, or deactivate, the enzymes and are therefore not suitable materials. Further, whilst silicone tubing has not been found to suffer from the same problem, silicon does tend to swell when used in the processes of the present invention which can lead to a fluctuation in the reaction conditions due to the consequential change in the residence time.

Any suitable circulation pump and substrate feed pump may be employed in the reactor loop. Examples of suitable circulation pumps include peristaltic, bellows, diaphragm, progressive cavity, piston, flexible linear, nutating disc, membrane, rotary lobe, flexible impeller, rotary vane, or any variable speed low shear type pump. Preferably, a peristaltic, flexible linear, nutating disc, or membrane pump is used. Not suitable as the circulating pump or substrate feed pump is a gear type pump.

For the efficient operation of the continuous membrane reactor of the present invention, the substrate feed pump operates at a greater speed than the circulation pump. For example, when the continuous membrane reactor is used with the processes of the present invention, the reaction performs most efficiently if the substrate feed pump is set to a speed about twenty times faster than the circulation pump.

In a preferred embodiment of the continues membrane reactor of the present invention, the reactor loop also comprises any of the following: a bubble trap, a pressure gage, a pH monitor, a heat exchanger, and a gate valve. In another preferred embodiment, the continuous membrane reactor comprises one or more substrate feed lines which comprise a substrate feed pump, and also more preferably comprise a check valve, a sterile filter, and a pressure gage. The addition of more than one feed lines allows for one to be used for feeding the substrates, and others other purposes, such as for sanitation purposes. The addition of a sterile filter to each of the feed lines aids in the removal of particles and microbes before they can enter into the reactor. Unwanted particles could block the pores of the membrane in the tangential flow filter unit, whilst microbes can kill certain enzymes. The addition of a heat exchanger can be used to maintain or vary the reaction temperature.

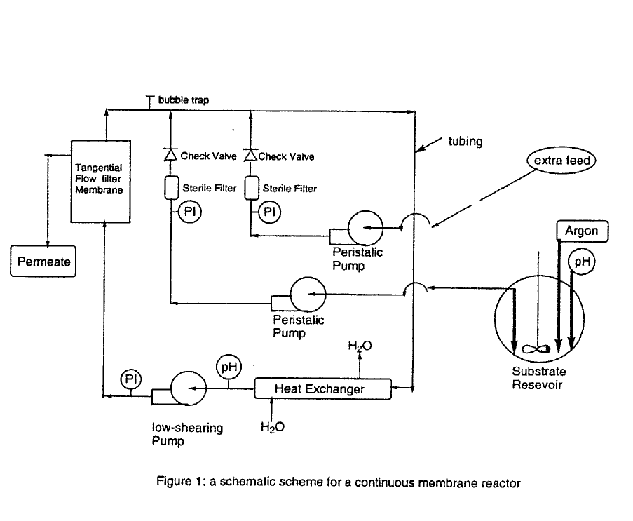

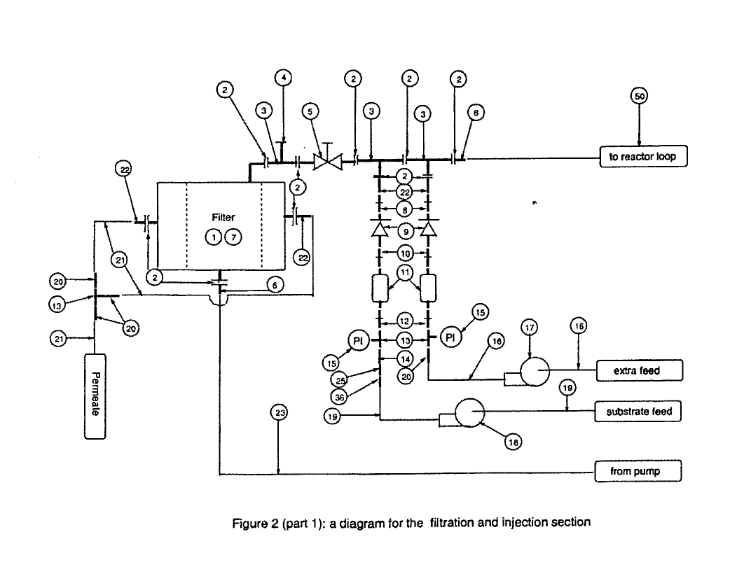

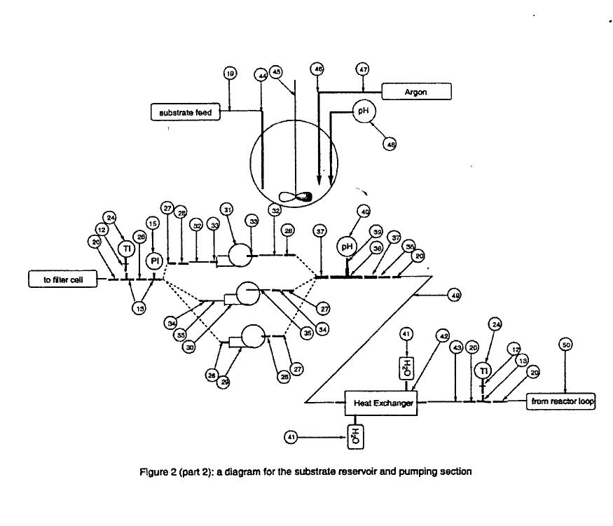

A preferred continuous membrane reactor is depicted in FIG. 1. A more preferred continuous membrane reactor of the present invention is depicted in FIG. 2, parts 1 and 2.

TABLE 1
list of the parts of the continuous membrane reactor depicted in FIG. 2

| | | Material of | | |
|---|---|---|---|---|
| Item | Description | Construction | Dimensions | Supplier |
| 1 | Tangential flow | 316 SS | | Waters |
| | filtration | | | Millipore |
| | membrane | | | |
| | housing; | | | |
| | Pellicon 2 | | | |
| 2 | Gasket & | Silicone & | 13/16″ × 1/16″ | Waters |
| | clamp | polypropylene | | Millipore |
| 3 | Sanitary tee | Polypropylene | 3″ × 1½″ × | Waters |
| | | | 7/16″ id | Millipore |
| 4 | Cap | Polypropylene | 1″ dia | Waters |
| | | | | Millipore |
| 5 | Gate valve | Polypropylene | 3″ × 7/16″ id | Waters |
| | | | | Millipore |
| 6 | Adapter | PVDF | 31/32″ × | Cole Parmer |
| | sanitary flange | | ½″ od | |
| | to hose barb | | | |
| 7 | Filtration | Regenerated | 0.1 sq M | Waters |
| | cartridge | cellulose | | Millipore |
| | PLCGC | | | |
| 8 | Compression | PVDF | 3/8″ od × ¼″ od | Cole Parmer |
| | reducing union | | | |
| 9 | One way check | PVDF/Kalrez | ¼″ od × 1½″ | Cole Parmer |
| | valve; 1 psi | | | |
| | seat pressure; | | | |
| | max 125 psi | | | |
| | back pressure | | | |
| 10 | Compression | PVDF | ¼″ od | Cole Parmer |
| | union | | | |
| 11 | Sterile filter; | PVDF/ | ¼″ od × 3 1/8″ | Cole Parmer |
| | 0.3 μM | borosilicate | | |
| | pore size | glass | | |
| 12 | Adapter | PVDF | ¼″ od × | Cole Parmer |
| | compression to | | ¼″ NPT | |
| | male NPT | | | |
| 13 | Tee female | | ¼″ NPT | Cole Parmer |
| | NPT | | | |
| 14 | Adapter male | PVDF | ¼″ NPT × | Cole Parmer |
| | NPT to | | ¼″ od | |
| | compression | | | |
| 15 | Pressure | 316 SS | ¼″ NPT; | Cole Parmer |
| | indicator 0-100 | | 2½″ face | |
| | psi; glycerin | | | |
| | filled;; | | | |
| | accuracy ± 3% | | | |
| 16 | Peristaltic | Peroxide | 0.19″ id | Cole Parmer |
| | tubing | cured silicone | | |
| 17 | Masterflex | 316 SS rollers | Flow 10-333 | Cole Parmer |
| | variable | | mL/min (with | |
| | speed drive | | item #16) | |
| | #77200-12 | | | |
| | with peristaltic | | | |
| | pump head | | | |
| | #77201-62 | | | |
| 18 | Masterflex | 316 SS rollers | Flow 0.2-20 | Cole Parmer |
| | console drive | | mL/min (with | |
| | with peristaltic | | item #19) | |
| | pump head | | | |
| | #77201-60 | | | |
| 19 | Peristaltic | Platinum cured | 0.06″ id | Cole Parmer |
| | tubing | silicone | | |
| 20 | Adapter hose | PVDF | 3/8″ od × | Cole Parmer |
| | barb to | | ¼″ NPT | |
| | male NPT | | | |
| 21 | Tubing | Peroxide | 3/8″ id | Cole Parmer |
| | | cured silicone | | |
| 22 | Adapter | PVDF | 31/32″ × | Cole Parmer |
| | sanitary flange | | 3/8″ od | |
| | to hose barb | | | |
| 23 | Pump line to | Viton | ½″ id | Cole Parmer |
| | filter | | | |
| 24 | “T” type | Teflon coated | ¼″ od | J-KEM |
| | thermocouple | 304SS | | |
| | and digital | | | |
| | readout | | | |
| 25 | Adapter tube | Teflon | ¼″ od × ¼″ 28 | Cole Parmer |
| | insert to | | UNF | |
| | female UNF | | | |
| 26 | Nipple | PVDF | ¼″ NPT | Cole Parmer |
| | male NPT | | | |
| 27 | Adapter | PTFE | ¼″ NPT × | Cole Parmer |
| | Bushing male | | ½″ NPT | |
| | NPT to female | | | |
| | NPT | | | |
| 28 | Adapter male | PVDF | 1/2″ NPT × ½ od | Cole Parmer |
| | PTO to | | | |
| | compression | | | |
| 29 | FMI ® Q | Ceramic/ | ¼″ piston dia. | Cole Parmer |
| | series variable | 316 SS | Flow 0-576 | |
| | speed valveless | | mL/min | |
| | metering pump | | | |
| | #QV & #R 405 | | | |
| 30 | Masterflex | 316 SS rollers | Flow 29-29000 | Cole Parmer |
| | variable speed | | mL/min (with | |
| | peristaltic | | item #35) | |
| | pump with | | | |
| | Easyload II | | | |
| | head | | | |
| | #77201-62 | | | |
| 31 | Milton Roy ® | Polypropylene/ | ¼″ id port Flow | Cole Parmer |
| | #C731-25T | Teflon/ceramic/ | 2.5-566 mL/min | |
| | variable speed | PFA/PTFE | | |
| | solenoid | | | |
| | diaphragm | | | |
| | pump | | | |
| 32 | Rigid tubing | Polyethylene | ½″ od × 3/8″ id | Cole Parmer |
| 33 | Adapter | Polypropylene | ½″ od × 7/8″ 12 | Cole Parmer |
| | compression to | | (custom) | |
| | pump check | | | |
| | valves | | | |
| 34 | Adapter male | PVDF | ¼″ NPT × | Cole Parmer |
| | NPT to | | 3/8″ od | |
| | hose barb | | | |
| 35 | Peristaltic | Peroxide | 0.38″ id | Cole Parmer |
| | tubing | cured silicone | | |
| 36 | Adapter male | PVDF | ¼″ UNF × | Cole Parmer |
| | UNF to hose | | 1/16″ od | |
| | barb | | | |
| 37 | Adapter | PTFE | ½″ NPT × | Cole Parmer |
| | bushing male | | 1″ NPT | |
| | NPT to female | | | |
| | NPT | | | |
| 38 | Tee female | PVDF | 1″ NPT | Cole Parmer |
| | NPT | | | |
| 39 | Adapter | PVDF | 1″ NPT × | Cole Parmer |
| | bushing male | | ¾″ NPT | |
| | NPT to female | | | |
| | NPT | | | |
| 40 | PH | PVDF/Viton/ | 2″ × ¾″ NPT | Cole Parmer |
| | indicator probe | glass | | |
| 41 | Cooling and/or | Water (or | N/A | N/A |
| | heating media | other media) | | |
| | flow | | | |
| 42 | Heat ex- | Glass | 38 cm internal × | San Diego |
| | changer—a | | 47 cm total | Glass Tech |
| | modified | | length; reactor | |
| | graham type | | volume 80 mL × | |
| | condenser with | | cooling volume | |
| | tube fittings on | | 510 mL | |
| | both ends | | | |
| 43 | Tubing | Viton | ½″ id | Cole Parmer |
| 44 | Rigid tubing | PTFE | ¼″ id × 5/16″ od | Cole Parmer |
| 45 | Mechanical | Glass/Teflon | As required | Chem Glass |
| | agitator | | | |
| 46 | Gas Diffuser | Glass | Coarse frit | Chem Glass |
| 47 | Tubing | Tygon R3603 | 5/16″ id | Fisher |
| 48 | PH probe with | Sealed gel | ½″ × 4½″ | Cole Parmer |
| | digital readout; | filled | | |
| | pn 59002-02 | | | |
| 49 | Tubing | Viton | ½″ id | Cole Parmer |
| 50 | Reactor main | Viton | ½″ id | Cole Parmer |
| | loop (tubing) | | | |

The following examples are provided merely for illustrative purposes of the present invention and are not to be read as limiting the scope of protection of the present invention, as defined by the appended claims.

### EXAMPLES

The following reaction schemes depict examples of the preparation of various compounds of the present invention, utilizing various processes of the present invention. In particular, the schemes depict example preparations set forth herein below.

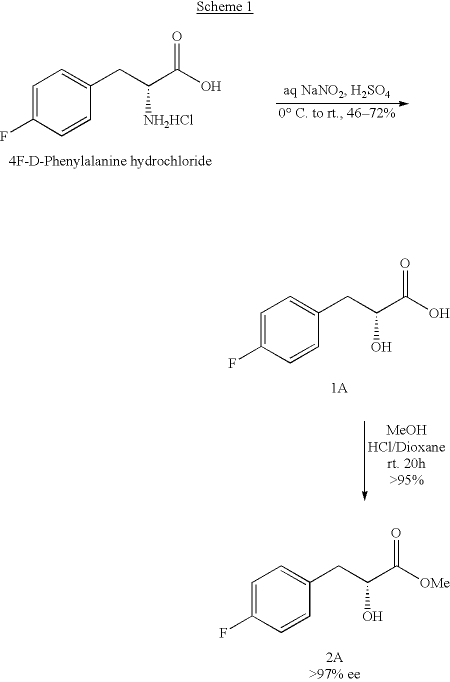

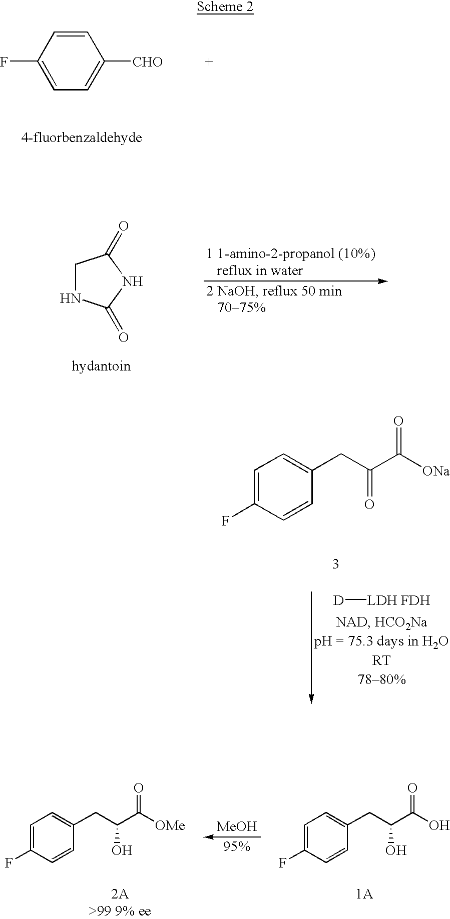

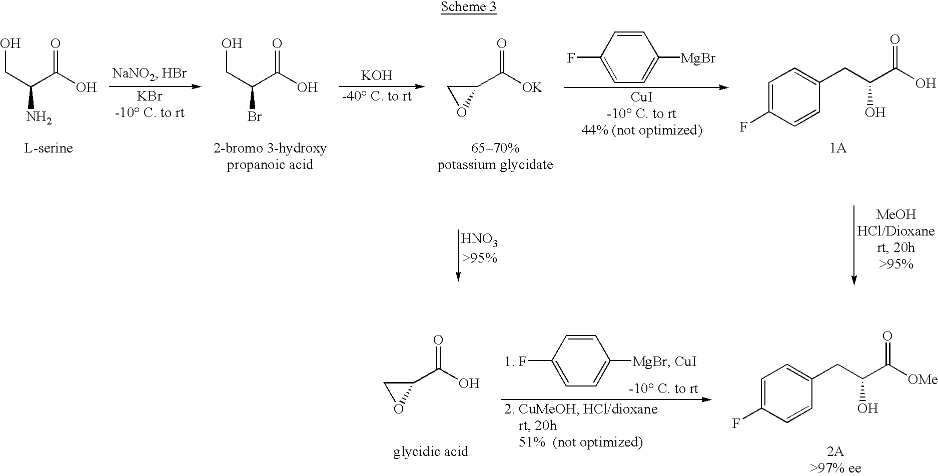

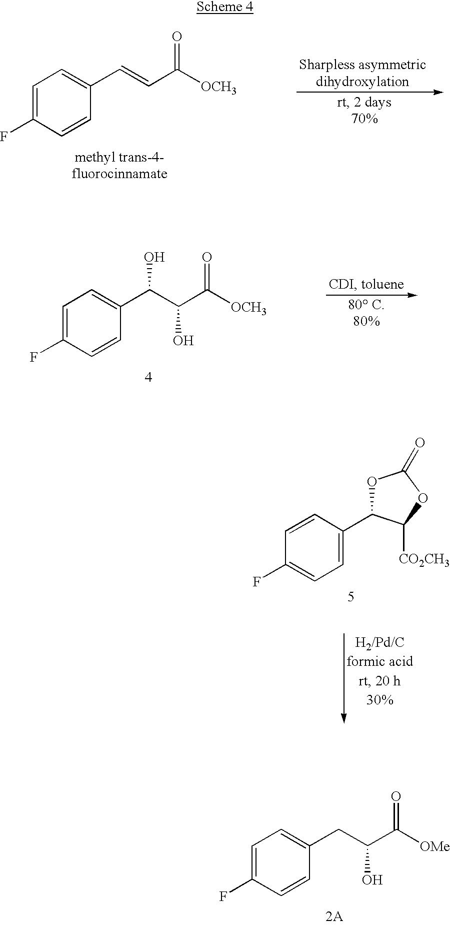

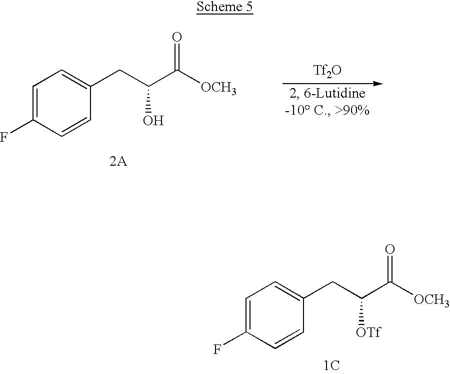

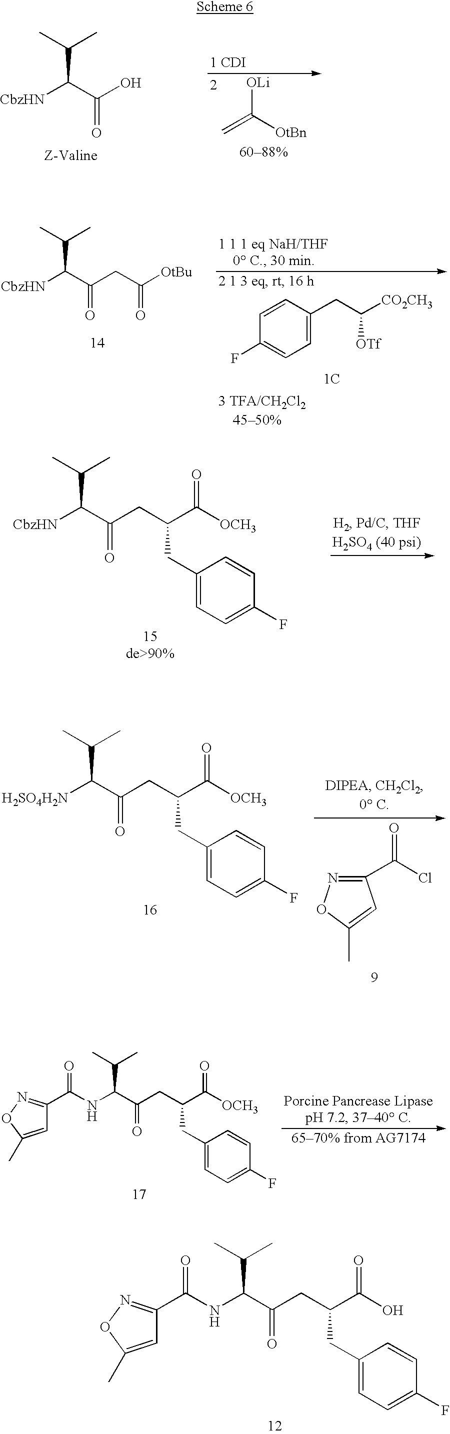

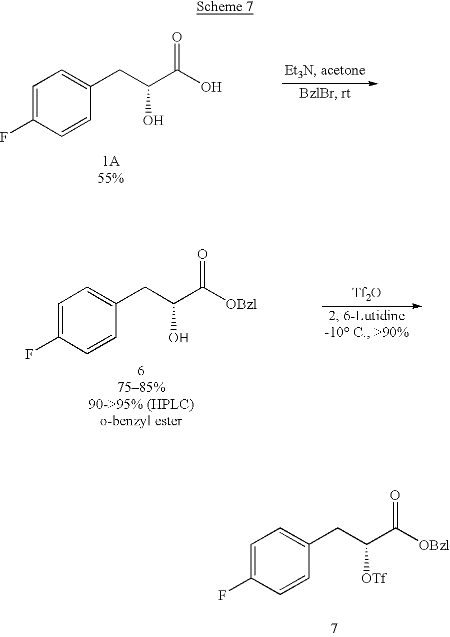

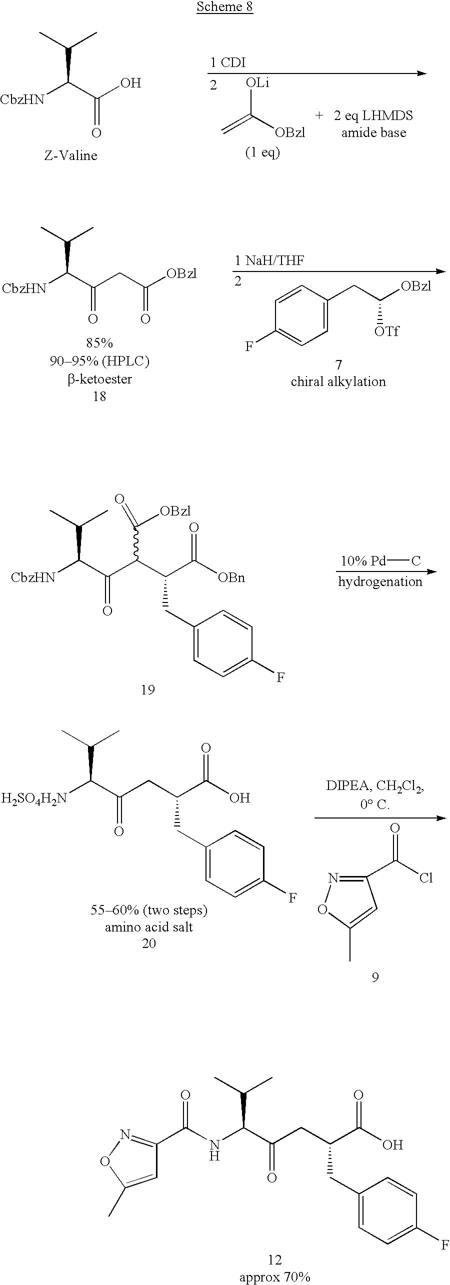

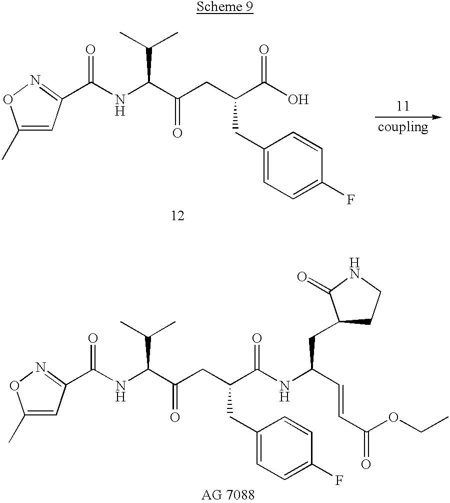

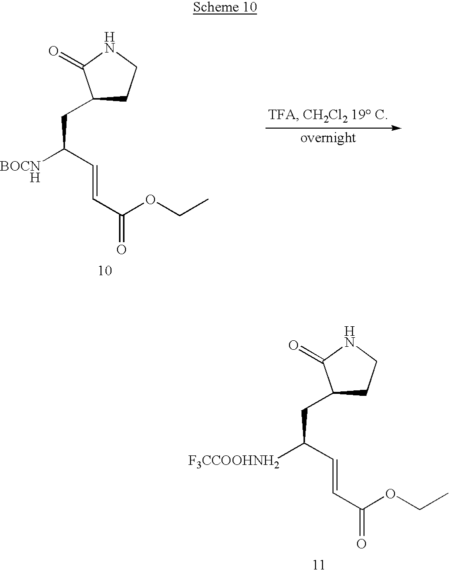

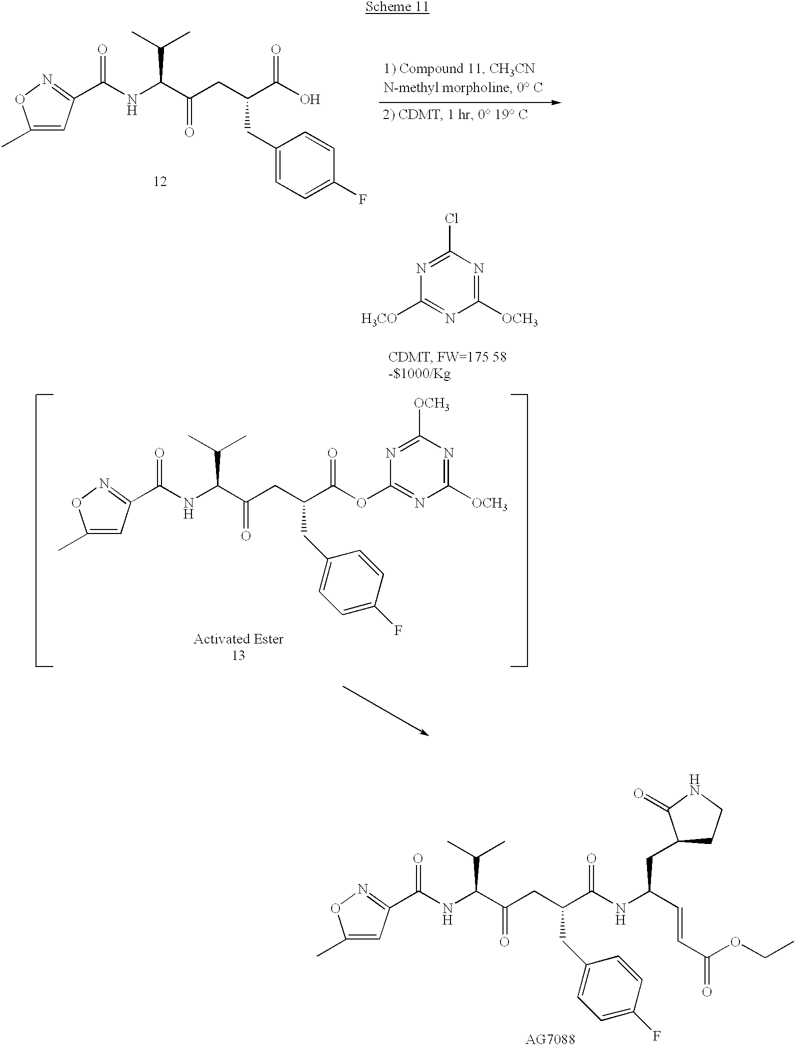

The following examples more fully describe the preparation of compounds of the present invention using the methods of the present invention.

### Example 1

Preparation of Compound 1A by Diazotization. (See Scheme 1 for the Structure of 1A.)

| Raw Material | Source | Amount | | MW | Moles |
|---|---|---|---|---|---|
| 4-fluoro-D-Phenyl- | 1443-057 | 380 | g | 219.5 | 1.73 |
| alanine hydrochloride | | | | | |
| 1 M H₂ SO₄ | Stock | 7.24 | L | — | 7.24 |
| (389 mL 98% sulfuric acid | | | | | |
| in 6.85 L water) | | | | | |
| 99.99% sodium nitrite | Aldrich | 477.5 | g | 69.0 | 6.92 |
| magnesium sulfate | Fisher | 100 | g | — | — |
| t-butyl methyl ether (MTBE) | Fisher | 3.6 | L | — | — |
| methylene chloride | Fisher | 1 | L | — | — |
| hexanes | Fisher | 2 | L | — | — |

Procedure:

In a 12 L reactor, 4-fluoro-D-phenylalanine hydrochloride (380 g) was dissolved in 7.24 L of 1 M sulfuric acid. The solution was cooled to −5° C. using acetone/ice. Then the solution was slowly charged with sodium nitrite (477.5 g dissolved in 730 mL water), keeping the temperature at or below 0° C. The addition time is typically 3 hours. The solution was held at 0° C. for 3 or more hours. It is important to maintain 0° C. during and after the addition for at least this stipulated time period. The reaction mixture was then warmed to ambient temperature over about 5 hours and held overnight. At this stage a white solid product, was seen floating in the reaction mixture. This product was extracted three times with MTBE (using 1.2 L MTBE per extraction, remembering to agitate the mixture vigorously each time for at least 15 min.) The organic extracts were dried with 100 g anhydrous magnesium sulfate, prior to filtration. The product was stripped to dryness (¹H NMR indicated at least 70% purity). At this stage, ˜380.5 g crude product was obtained. The crude solid 1A was taken up in 1 L methylene chloride and 2 L hexanes and brought to reflux (42° C.). It was held for 2 hours at reflux with good agitation, before being cooled to ambient temperature. It was then held for a further 2 hours at ambient temperature with stirring. After filtration, the cake was rinsed with 2:1 hexanes/methylene chloride. The reaction yielded 148 g (46%) of compound 1A; Chiral HPLC purity >97%; ¹H NMR (CD₃OD) δ 7.25-7.00 (m, 4H), 4.50 (AB quartet, J=8 Hz, J=4 Hz, 1H),3.15 (dd, J=14 Hz, J=4 Hz, 1H) 2.95 (dd, J=14 Hz, J=8 Hz, 1H).

### Example 2

Preparation of 1A by Enzymatic Reduction:

Step A—Preparation of compound 3. (See scheme 2 for structure of 3.)

| Raw material | Source | Amount | | MW | Moles |
|---|---|---|---|---|---|
| 4-fluorobenzaldehyde | Aldrich | 115.92 | g | 124.11 | 0.934 |
| hydantoin | Aldrich | 93.53 | g | 100.08 | 0.934 |
| 1-amino-2-propanol | Aldrich | 7.01 | g | 75.11 | 0.0934 |
| sodium hydroxide | Fisher | 187 | g | 40.00 | 4.68 |
| sodium chloride | Fisher | 108.9 | g | 58.44 | 1.86 |
| Conc. HCl (37%) | Fisher | 311 | mL | — | — |

Procedure:

A mixture of 4-fluorobenzaldehyde, hydantoin, and 1-amino-2-propanol (10%) in water (235 mL) was refluxed for 4 hours (130-135° C.). The mixture was charged with 935 g of 20% hot aqueous sodium hydroxide solution (187 g of NaOH, 4.68 mol) and refluxed for 50 minutes. The mixture was then cooled to 0° C. and charged with 108.9 g of sodium chloride. The pH of the solution was adjusted to about 8.5 using concentrated HCl (37%, ca. 311 mL) at 0° C., before being filtered. The mother liquor was left to stand overnight and filtered again. The precipitates were combined and washed with methanol (about 5L) to get a HPLC purity of >80%. (Note: this salt is pure enough for subsequent enzymatic reaction, but higher purity can be obtained by washing with extra amounts of methanol). The precipitate was dried under a house vacuum to get a white monohydrated sodium salt 3: yield 70-75%. Analysis calculated for C₉H₆O₃Fna. H₂O: C, 48.66; H, 3.63. Found: C, 48.64; H, 3.74. ¹H NMR (D₂O) δ7.02-7.19 (m, 4H), 4.72 (s, 2H) (Note: this salt should be stored in a refrigerator to prevent decomposition.)

Step B—Preparation of 1A from 3

Either the MEEC method (procedure B 1) or coimmobilization method (procedure B2) may be used to prepare 1A.

Procedure B1: Preparation of 1A using the MEEC method

| Raw material | Source (catalog #) | Amount | | MW | Moles |
|---|---|---|---|---|---|
| Compound 3 | — | 11.1 | g | 222 | 0.05 |
| D-lactate | Sigma (L 9636) | 1900 | U | — | — |
| dehydrogenase | | | | | |
| (D-LDH) | | | | | |
| formate dehydrogenase | Sigma (F 8649) | 125 | U | — | — |
| (FDH) | | | | | |
| NAD | Sigma (N 7004) | 334 | mg | 663.4 | 0.0005 |
| Sodium formate | Sigma (S 2140) | 10.25 | g | 68.01 | 0.15 |
| mercaptoethanol | Sigma (M 6250) | 39 | mg | 78.13 | 0.0005 |
| Trizma | Sigma (T 6666) | 400 | mg | 157.6 | 0.0025 |
| hydrochloride | | | | | |
| EDTA | Sigma (E 1644) | 186 | mg | 372.2 | 0.0005 |

| | | | | |
|---|---|---|---|---|
| DL-dithiothreitol | Sigma (D 5545) | — | — | — |
| Dialysis membrane | VWR | — | — | — |
| (MWCO 12,000- | (25218-435) | | | |
| 14,000) | | | | |

Procedure:

3, sodium formate, mercaptoethanol, Trizma hydrochloride and EDTA were dissolved in deionized water (500 mL) and the solution was degassed with argon for about 30 minutes. The solution was adjusted to a pH of about 7.5 using NaOH (1.0 M), and NAD (1%) added. Four dialysis tubing's (ca.4 cm long each) were rinsed with deionized water and one of the ends of each were tied off with thread. FDH and D-LDH were dissolved in an 8-mL aliquot of the reaction mixture and transferred to the 4 tubing's (ca. 2 mL each) using an Eppendorf pipette. The other ends of the tubing's were tied and suspend in the reaction mixture. (Note: care was taken to exclude as much air as possible and make sure there was no leakage). Argon was gently bubbled through the solution to remove CO₂. The mixture was then stirred at room temperature for 3 days keeping the pH at 7.5±0.1 by pH-stat. controlled addition of 1 M HCl (>95% conversion by HPLC). The dialysis tubing's were then removed. The stirring was continued for about 6 hours in 100 mL of 50 mM Tris buffer (pH 7.5, 5 mM dithiothreitol). (Note: the enzyme-containing bags can be reused by storage at 4° C. in 50 mL of 5 mM tris buffer (pH 7.5, 5 mM dithiothreitol)). The aqueous layers were combined and the solution adjusted to a pH of 3.0 by slowly adding concentrated HCl. The solution was extract with MTBE (50×4 mL), dried with MgSO₄ and concentrated to a crude oil. The oil was solidified in 250 mL of hexanes/methylene chloride (2:1) and filtered. The filtrate was then concentrated and solidified again in 50 mL of hexanes/methylene chloride (2:1). The white solids were combined and dried under house vacuum to yield a white solid 1A: yield 7.2-7.4 g (78-80%); HPLC purity >95%.

Procedure B2: Preparation of 1A using the Coimmobilization Method

This procedure was carried out in 4 steps. The first step was the preparation of N-acryloxysuccinimide. The second was the preparation of PAN 500 by a radical copolymerization. The third was a coimmobilization of FDH and D-LDH. The last step was an enzymatic reduction of the a-keto acid sodium salt 3 to give 1A.

Step 1: Preparation of N-acryloxysuccinimide

| Raw material | Source | Amount | | MW | Moles |
|---|---|---|---|---|---|
| Acryloyl chloride | Aldrich | 100 | g | 90.51 | 1.10 |
| N-hydroxysuccinimide | Aldrich | 115 | g | 115.10 | 1.00 |
| triethylamine | Aldrich | 110 | g | 101.19 | 1.09 |
| 2,6-di-tert-butyl-4- | Aldrich | 50 | mg | 220.36 | 0.00023 |
| methylphenol (BHT) | | | | | |

Procedure:

N-hydroxysuccinimide and triethylamine were dissolved in 1.5 L of chloroform at 0° C. Acryloyl chloride was added dropwise over a 20 minute period and stirred for an additional 20 minutes at 0° C. The solution was washed with ice-cold 800 mL portions of water and saturated brine, then dried with MgSO₄ and filtered. 50 mg of BHT was added to the chloroform solution, and concentrated to a volume of 300 mL and filtered. Slowly, 30 mL ethyl acetate and 200 mL hexanes were added to the solution while stirring, before being left to stand at 0° C. for 2 hours. The white solid produced was filtered and washed first with ice-cold 100 mL hexanes/ethyl acetate (4:1), then with 100 mL hexanes/ethyl acetate (9:1), and finally with hexanes (100 mL ×2). (Note: this material is pure enough to be used for the preparation of PAN 500 disclosed below). The crystals were dried under house vacuum to yield N-acryloxysuccinimide; yield 115 g (68%); mp 68-70° C. ¹H NMR (CDCl₃) δ 6.0-7.0 (m, 3H), 2.85 (s, 4H); FTIR (Nujol) 1800, 1775, 1735, 1260, 995, 870 cm⁻¹.

Step 2: Preparation of PAN 500

| Raw material | Source | Amount | | MW | Moles |
|---|---|---|---|---|---|
| N-acryloxysuccinimide | — | 30 | g | 169.1 | 0.178 |
| acrylamide | Aldrich | 275 | g | 71.08 | 3.85 |
| AIBN | Aldrich | 1.75 | g | 164.21 | 0.011 |
| THF | Fisher | 2.5 | L | — | — |

Procedure:

Acrylamide, N-acryloxysuccinimide, AIBN, and THF (2.5 L) were charged in a 5 L flask. The solution was degassed with argon for 30 minutes under vigorous stirring and then refluxed at 50° C. under argon for 24 hours. (Caution: this reaction is exothermic during the first 1-2 hours). It was then charged with 1 L of THF and stirred for 10 minutes. The precipitate formed was filtered off and washed with THF (1 L×4). The product was dried under house vacuum to yield PAN 500: yield ˜304 g of a very fluffy, white powder. FTIR (Nujol) 3340, 3200, 1730, 1660, 1210, 1070 cm⁻¹. (Note: this polymer should be stored in a drying dessicator).

Step 3: coimmobilization of FDH and D-LDH

| Raw material | Source | Amount | | MW | Moles | |
|---|---|---|---|---|---|---|
| triethylenetetra- | Aldrich | — | | 146.24 | — | |
| mine (60%, TET) | | | | | | |
| MgCl₂ 6H₂O | Sigma | 50 | mg | 203.3 | 0.24 | mmol |
| Sodium pyruvate | Sigma | 50 | mg | 110.0 | 0.45 | mmol |
| NADH | Sigma | 50 | mg | 709.4 | 0.07 | mmol |
| sodium formate | Sigma | 306 | mg | 68.01 | 4.5 | mmol |
| NAD | Sigma | 111 | mg | 663.4 | 0.17 | mmol |
| FDH | Sigma (F 8649) | 200 | U | — | — | |
| D-LDH | Sigma (L 2395) | 5000 | U | — | — | |
| Hepes | Sigma (H 9897) | — | | — | — | |
| DL-dithiothreitol | Sigma (D 5545) | — | | — | — | |
| ammonium | Sigma | 1.32 | g | 132.1 | 0.01 | mol |
| sulfate | | | | | | |

Procedure:

5000 U of commercially available D-LDH in 3.2M (NH₄)₂SO₄ was centrifuged at 4° C. for 10 minutes. The resulting precipitate was dissolved in 3 mL of 0.3 M Hepes buffer (pH 7.5), and dialyzed against 500 mL of 50 mM deoxygenated Hepes buffer (pH 7.5) at 4° C. under argon overnight with stirring. This solution was charged with 13.0 g of PAN 500 by adding it to a 500-mL beaker to which was added 42 mL of 0.3 M Hepes buffer (pH 7.5) containing magnesium chloride, sodium pyruvate, NADH, NAD and sodium formate. The mixture was stirred vigorously for 1 minute before DL-dithiothreitol (650 μL, 0.50 M) and TET (5.53 mL, 0.50 M) were added to the mixture. The mixture was then stirred for 1 minute before D-LDH and FDH were added. (Note: the mixture gelled after ca. two additional minutes of stirring). The gel was kept at room temperature for 1 hour before ca. 200 mL of 5 mM Hepes buffer (pH 7.5, containing 1.32 g (NH₄)₂SO₄) was added. The gel was broken in a Waring blender at low speed for 3 minutes and then at high speed for 30 seconds. The gel particles were separated by centrifugation, washed with 20 mL of 50 mM Hepes buffer (pH 7.5), and separated again by centrifugation.

Step 4: Preparation of 1A using coimmobilization

| Raw material | Source (catalog #) | Amount | | MW | Moles |
|---|---|---|---|---|---|
| compound 3 | — | 11.10 | g | 222 | 0.050 |
| PAN coimmobilized | — | — | | — | — |
| D-LDH and FDH | | | | | |
| NAD | Sigma (N 7004) | 167 | mg | 663.4 | 0.00025 |
| sodium formate | Sigma (S 2140) | 4.10 | g | 68.01 | 0.060 |
| mercaptoethanol | Sigma (M 6250) | 19.5 | mg | 78.13 | 0.00025 |
| Trizma | Sigma (T 6666) | 150 | mg | 157.6 | 0.00095 |
| hydrochloride | | | | | |

| | | | | |
|---|---|---|---|---|
| DL-dithiothreitol | Sigma (D 5545) | — | — | — |

Procedure:

3, sodium formate, mercaptoethanol, and Trizama hydrochloride were dissolved in deionized water (500 mL) and the solution degassed with argon for 30 minutes. The solution was adjusted to a pH of about 7.5 and NAD (1%) added. The coimmobilized FDH and D-LDH PAN gel were added. Argon was gently bubbled through the solution to remove CO₂, and the mixture was then stirred at room temperature for 5 days at a pH of about 7.5±0.1 by the pH-stat. controlled addition of 1 M HCl (>91% conversion by HPLC). (Note: the use of excess sodium formate leads to a shorter reaction time, see MEEC method.) The enzyme-containing gels were removed by centrifugation, and washed twice with 50 mL portions of degassed water. (Note: the enzyme-containing gels can be reused by storage at 4° C. in 50 mL of 5 mM Tris buffer (pH 7.5, 5 mM dithiothreitol)).

The aqueous layers were combined and the solution adjusted to a pH of 3.0 by the slow addition of concentrated HCl. The product was extract with MTBE (50×4 mL), dried with MgSO₄ and concentrated to yield a crude oil product. The oil was, solidified in 250 mL hexanes/methylene chloride (2:1) and filtered. The filtrate was concentrated and solidified again in 50 mL hexanes/methylene chloride (2:1). The white solids were combined and dried under house vacuum to yield a white solid product, compound 1A: yield 7.2 g (78%); HPLC purity >95%.

### Example 2A

Preparation of 1A by Enzymatic Reduction Using the Continuous Membrane Reactor of the Present Invention:

Step A—Preparation of compound 3.

| Raw material | equiv. | Moles | F.W. | Amount | |
|---|---|---|---|---|---|
| p-fluoro- | 1.0 | 20 | 124.11 | 2482 | g |
| benzladehyde (I) | | | | | |
| Hydantoin | 1.0 | 20 | 100.08 | 2002 | g |
| 1-amino-2-propanol | 0.1 | 2 | 75.11 | 150 | g |
| Water | 0.25 L/mol of (I) | — | — | 5.0 | L |
| Sodium Hydroxide | 5.0 | 100 | 40.00 | 4000 | g |
| (pellets) | | | | | |
| Water | 5.0 L/kg of NaOH | — | — | 20.0 | L |
| Sodium chloride | 2.0 | 40 | 58.44 | 2338 | g |
| (granular) | | | | | |
| HCl (conc.) | 5.0 | 100 | 36.46 | 8.26 | L |
| Methanol | 3.33 L/mol of (I) | — | — | 66.7 | L |

Procedure:

To a 50 L reactor equipped with a temperature probe, reflux condenser, agitator and cooling coils was added 2.482 kg of p-fluoro-benzladehyde, 2.002 kg hydantoin, and 150 g of 1-amino-2-propanol in water. The resulting mixture was heated and refluxed for about 10 hours. The solution was monitored for the disappearance of p-fluoro-benzladehyde by both HPLC (254 nm) and by ¹H NMR (the movement of the aldehyde proton from 10 ppm to 7.2 ppm.) The reaction yielded a yellow slurry. HPLC analysis of the yellow slurry appeared to show only a 35% conversion, however, ¹H NMR showed about a 90% conversion. It is thought that the HPLC method indicated an inaccurately low conversion due to the strong chromophore of benzaldehyde.

A separate sodium hydroxide in water solution was then prepared which was heated to about 98° C. This solution was then carefully added to the yellow slurry. The reaction mixture was then refluxed for about 3 hours, before being allowed to cool to room temperature. Again the reaction mixture was monitoring by HPLC (254 nm) for the complete disappearance of the condensed intermediate peak. The resulting reaction mixture was in the form of a transparent orange/yellow solution.

Once the reaction mixture was cooled to about 20° C.±5° C., sodium chloride was added and the reaction mixture agitated. While maintaining the coolant flow a pH probe was inserted and concentrated hydrochloric acid added to adjust the pH to between about 8.0-8.5. Whilst adjusting the pH, the reaction temperature was maintained at a temperature under about 30° C. by regulating the rate of acid addition.

After about 4 hours, the resulting reaction mixture, in the form of a pale yellow slurry, was filtered through a table top buchner funnel fitted with #1 filter paper. The wet cake was then washed by returning it to the 50 L reactor, adding to it about 33.35 L methanol and then stirring it for 15 minutes. The solids are again filtered off using a buchner funnel and the wet cake washed using the same procedure again using about 33.35 L methanol.

The resulting washed solids were then dried in an oven at room temperature under house vacuum for about four days to yield a white to off-white solid, compound 3. The product was >80% pure by HPLC (254 nm) with a yield of about 75%. ¹H NMR (D₂O) δ 7.02-7.19 (m 4H), 4.72 (s, 2H).

Step B—Preparation of compound 1A using the
continuous membrane reactor of the present invention.

| | | Milli- | | | |
|---|---|---|---|---|---|
| Raw material | equiv. | moles | F.W. | amount | |
| P2-hydroxy acid, 3 | 1.0 | 1350 | 222.14 | 299.9 | g |
| Mercaptoethanol | 1.0 mM | 6.75 | 78.13 | 527 mg + 195 mg | |
| Ammonium formate | 4.0 | 5400 | 63.06 | 340.5 | g |
| Water | 5.0 L/mol | — | — | 6.75 | L |
| | of 3 | | | | |
| β-NAD | 0.01 | 13.5 | 663.4 | | |
| Formate | 20,000 U/ | — | — | 4,800 | units |
| Dehydrogenase | Liter of | | | | |
| | reactor | | | | |
| Lactic | 400,000 U/ | — | — | 96,000 | units |
| Dehydrogenase | Liter of | | | | |
| | reactor | | | | |
| HCl (conc.) | — | — | 36.46 | A.R. ˜450 | mL |
| MTBE | 6.5 L/mol | — | — | 8.78 | L |
| | of 3 | | | | |
| Brine | 1.1 L/mol | — | — | 1.49 | L |
| | of 3 | | | | |
| Magnesium sulfate | | — | | | |
| Dichloromethane | 1.46 L/mol | — | — | 2.00 | L |
| | of 3 | | | | |
| Hexanes | 2.93 L/mol | — | — | 3.96 | L |
| | of 3 | | | | |

Procedure:

Ensuring the continuous membrane reactor (240 mL) was assembled as set forth in the present disclosure as shown in FIG. 2, parts 1 and 2, the reactor was washed with 0.02% v/v solution of peracetic acid in water until a total of 2.5 liters had been removed from the permeate port. Then, a solution of 2.5 L of 0.2 μM filtered water and 0.1 mM mercaptoethanol (195 mg) was prepared and used to flush the reactor.

To a 12 L round bottom flask with overhead stirrer, pH meter and a gas diffuser was added 6.75 L of water that had been filtered through a 0.2 μM or finer filter. The flask was then purged with argon for at least about 30 minutes.

Compound 3 was then added to the 12 L flask and dissolved into the degassed water therein, along with ammonium formate and mercaptoethanol. Maintaining the argon purge, the resulting solution was stirred until all the solids had dissolved. Once dissolved, the pH of the solution was adjusted to about 7.0 using 1 N sodium hydroxide. β-NAD was then added to the reaction solution, and stirred to dissolve the solids. The pH was then adjusted to about 6.2 to yield a substrate solution.

The enzymes, Formate Dehydrogenase and Lactic Dehydrogenase, were then dissolved using 100 mL of the substrate solution before they were added to the reactor by pumping them in through the substrate feed line. The rate of pumping began at about 1.0 mL/min. Care was taken to maintain the pH of the substrate solution at about 6.2. The effluent (or permeate) was monitored for conversion by HPLC (254 nm). The pH of the effluent was also checked frequently, which also helped to monitor the conversion (should be pH=7.3-7.4. The feed rate (the rate of pumping) was adjusted as necessary to increase the conversion and/or the throughput as it was needed.

Once the substrate had been completely fed through reactor the resulting permeate was worked up by acidification to a pH of about 3.0, using concentrated hydrochloric acid. The resulting solution was then extraction with MTBE, divided into three separate portions. The MTBE extracted solution was washed with brine, dried over magnesium sulfate, filtered and concentrated on rotovapour to yield a yellow oil.

To the oil was added 810 mL dichloromethane until all the oil had dissolved. Slowly, 1.62 L hexanes were added to the solution, and the solution heated to reflux, cooled to 10° C. whilst being agitated.

A solid product was then filtered off and washed as a cake with 1.2 L of a 2:1 hexanes:dichloromethane solution. The washed solids were then dried under house vacuum for three days at room temperature to yield a white powder. The yield was about 70% (purity 91% by HPLC) with a productivity of the reactor of 280 g/(d×L). ¹H NMR (CD₃Cl) δ 7.25-7.00 (m, 4H), 4.50 (AB quartet, J=4, 8 Hz, 1H), 3.15 (dd, J=4, 14 Hz, 1H), 2.95 (dd, J=8, 14 Hz, 1H). The enantiomeric purity of the corresponding methyl ester of the sample was >99.99% (Chiralpak AS, 4.6×250 mm, 10.0 μm).

### Example 2B

Preparation of 1A from 3 Using the Continuous Membrane Reactor of the Present Invention:

| | | Milli- | | | |
|---|---|---|---|---|---|
| Raw material | equiv. | moles | F.W. | amount | |
| Ketoacid salt, 3 | 1.0 | 5400 | 222.14 | 1.2 | kg |
| Mercaptoethanol | 1.0 mM | 27 | 78.13 | 2.1 | g |
| Ammonium formate | 4.0 | 21600 | 63.06 | 1.36 | kg |
| Water | 5.0 L/mol | — | — | 27 | L |
| | of 3 | | | | |
| β-NAD | 0.01 | 54 | 663.4 | 35.82 | g |
| Formate | 20,000 U/ | — | — | 30,900 | units |
| Dehydrogenase | Liter of | | | | |
| | reactor | | | | |
| Lactic | 400,000 U/ | — | — | 618,000 | units |
| Dehydrogenase | Liter of | | | | |
| | reactor | | | | |
| HCl (conc.) | — | — | 36.46 | A.R. 1650 | mL |
| MTBE | 6.5 L/mol | — | — | 35.1 | L |
| | of 3 | | | | |
| Brine | 1.1 L/mol | — | — | 5.94 | L |
| | of 3 | | | | |
| Magnesium sulfate | | — | | | |
| Dichloromethane | 1.46 L/mol | — | — | 7.88 | L |
| | of 3 | | | | |
| Hexanes | 2.93 L/mol | — | — | 15.8 | L |
| | of 3 | | | | |

Procedure:

Once a continuous membrane reactor of the present invention had been assembled as shown in FIG. 2, parts 1 and 2, having a capacity off 1.545 L, the reactor was washed with 0.02% v/v solution of peracetic acid in 0.2 μM filtered water, i.e., water that had been filtered through a 0.2 μM or finer filter, until about 15 L of the solution had been removed from the permeate port. The reactor was then flushed with 15 L of 0.2 μM filtered water. 15 L of a solution of 0.2 μM filtered water and 0.1 mM mercaptoethanol was prepared and used to flush the reactor.

To a 22 L round bottomed flask fitted with an overhead stirrer, pH meter and gas diffuser was added 9.0 L 0.2 μM filtered water. The flask was then purged with argon for at least 30 minutes. 400 g of 3 was dissolved into the degassed water along with ammonium formate and mercaptoethanol. Maintaining the argon purge, the solution was stirred until all the solids had dissolved. Once the solids had dissolved, the pH of the solution was adjusted to a pH of about 6.26 using 1 N HCl. Then β-NAD was added to the solution and the solution stirred until it dissolved. The resulting substrate solution was kept at a pH of about 6.26.

The enzymes (Formate Dehydrogenase and Lactic Dehydrogenase) were then dissolved in 600 mL of the substrate solution. The substrate solution containing the enzymes was then put into the reactor by feeding the solution through the substrate feed line of the reactor.

The remainder of the substrate mixture was then pumped into the reactor at a rate of 7.6 mL/minute. Care was taken to maintain the pH of the substrate solution at about 6.26, and to maintain a slight argon purge. The effluent was monitored for conversion by HPLC (254 nm). Also the pH of the effluent was checked often which also helped to monitor the conversion (pH should be 7.3-7.4). Note: The feed rate may be adjusted as necessary to vary the conversion or throughput rate as desired. The conversion was found to be 90-95% by HPLC.

One the initial solution containing 400 g of 3 had been fed into the reactor, another substrate solution containing 400 g of 3, prepared in the same manner as described above was prepared and pumped into the reactor. This was repeated until a total of 1.2 kg of 3 had been used. In the overall 1.2 kg run, no further enzymes were used by the reactor, and the conversion of 3 to 1A was found to be greater than 90% by HPLC.

### Example 3

Preparation of 2: (See Scheme 1 for the Structures of 1A and 2A)

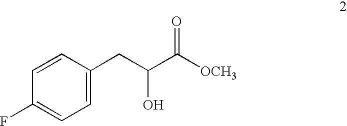

| Raw material | Source | Amount | | MW | Moles |
|---|---|---|---|---|---|
| 1A | 1443-111 | 144 | g | 184.2 | 0.781 |
| methanol | Fisher | 950 | mL | — | — |
| 4M HCl/Dioxane | Aldrich | 18 | mL | — | — |
| hexanes | Fisher | 300 | mL | — | — |

Procedure:

144 g of compound 1A was stirred in 950 mL methanol and 18 mL 4 M HCl/dioxane at ambient temperature for 20 hours. The completion of the reaction was confirmed by HPLC. Once complete, the solvents were stripped off under vacuum. The concentrated product (oily at this stage) was agitated vigorously with an overhead stirrer, while 300 mL of hexanes was added slowly. Agitation was maintained for 30 minutes. Compound 2A was a powdery solid at this stage. It was cooled to 10° C. and filtered to yield a solid product. Further, the filtrate upon concentration also yielded 4-5 g of clean product also. Upon confirming by HPLC that the concentrated filtrate was indeed the clean product, both solids were combined and dried under vacuum at room temperature (note: alternatively MTBE/aq. saturated bicarbonate wash can be employed to remove acidic impurities carried over from 1A). Yield of compound 2, 141 g (95%); Chiral HPLC purity >97% ee. ¹H NMR (CDCl₃) δ 7.25-7.00 (m 4H), 4.50 (AB quartet, J 8 Hz, J=4Hz, 1H), 3.82 (s, 3H), 3.15 (dd,J=13 Hz, J=4Hz, 1H), 2.95 (dd, J=14 Hz, J =7 Hz, 1H), 2.85 (br.s, 1H). (Note: enzymatic methods lead to compound 2A with >99.9% ee).

### Example 4

Preparation of 1C: (See Scheme 5 for the Structures of 2A and 1C.)

| Raw material | Source | Amount | | MW | Moles |
|---|---|---|---|---|---|
| 2A | — | 200 | g | 198.2 | 1.01 |
| MTBE | Fisher | 4 | L | — | — |
| 99.99% Triflic anhydride | Aldrich | 484 | g | 202.1 | 1.71 |
| 2,6 Lutidine | Aldrich | 184 | g | 107.1 | 1.71 |
| 1M Citric acid | Stock | 2 × 1 | L | — | — |
| Satd. Sodium bicarbonate | stock | 2 × 1 | L | — | — |
| Anhyd. Magnesium sulfate | — | 150 | g | — | — |

Procedure:

200 g of compound 2A was dissolved in 4L MTBE under nitrogen and cooled to −10° C. The triflic anhydride was added via an addition funnel over 15 minutes, followed by the slow addition of 2,6-lutidine via an addition funnel wherein the internal temperature was kept below 3° C. The mixture was then stirred for one hour at 0° C. before 1.9L water was added while stirring. The solution was stirred for an additional 15 minutes. The top organic layer was then separated and washed twice with 1L (1M) citric acid and then twice with 1L saturated sodium bicarbonate solution. It was then dried over anhydrous magnesium sulfate, filtered over celite, and stripped to an oil under vacuum to yield compound 1C. Yield of 1C, 340 g (95%); ¹H NMR (CDCl₃) should indicate that the compound is greater than 95% pure. (Note: If the conversion is not complete, repeat the above steps depending on the extent of conversion.) After this rework operation had been carried out the isolated product conformed to >95% (¹H NMR) purity. This triflate, compound 1C, should be stored cold to prevent decomposition.

### Example 5

Procedure for the Preparation of 14: (See Scheme 6 for the Structures of the Compounds Referred to in Examples 5-9.)

| Raw material | Source | Amount | | MW | Moles |
|---|---|---|---|---|---|
| Z-L-Valine | Calbiochem | 200 | g | 251 | 0.796 |
| Carbonyldiimidazole | Aldrich | 135 | g | 162 | 0.836 |
| (CDI) | | | | | |
| Anhydrous THF | Fisher | 3.3 | L | — | — |
| 1M Lithium | Aldrich | 2.78 | L | 1 M soln. | 2.78 |
| bistrimethylsilylamide | | | | | |
| in THF (LHMDS in | | | | | |
| THF) | | | | | |
| t-Butyl acetate | Aldrich | 355 | g | 116 | 3.06 |
| 1M HCl | Stock | 10 | L | — | — |
| MTBE | Fisher | 8 | L | — | — |
| Sat. Sodium bicarbonate | Stock | 4 | L | — | — |
| Brine | Stock | 2 | L | — | — |
| Anhydrous Magnesium | Fisher | 300 | g | — | — |
| Sulfate | | | | | |

Procedure:

200 g Z-L-Valine was stirred with CDI in 3.3L THF under a nitrogen stream for about 1 hour at room temperature. After an hour, the imidazolide formation should be complete. In a 12L reactor, the reaction mixture was charged with 2.78L 1M lithium bistrimethylsilylamide solution in THF under nitrogen, then cooled to −70° C. t-Butyl acetate (410g) was then added slowly over a period of about 1 hour keeping the temperature below −60° C. The reaction mixture was then stirred for 30 minutes at −60° to −70° C. The anhydride prepared in the step above was taken in an addition funnel and slowly added to the enolate, under nitrogen with good stirring, keeping the internal temperature at or below −60° C. Once the addition was complete, the reaction mixture was stirred at −60° C. for 1 hour. It was then charged with 4.0L 1M HCl, slowly, with rigorous stirring, keeping the internal temperature below −50° C. Higher temperatures during quenching causes racemization. 200 mL concentrated 12M HCl was added to adjust the pH to between 6-7.5. A lot of solid fell out of solution—mostly imidazole and trapped organic impurities and amine salts. These solids were filtered off over celite. Because warmer temperatures tend to dissolve impurities, the filtration was carried out cold and rapidly over celite. The solids were washed with 4L MTBE. The filtrate was diluted with 2L MTBE and 1M HCl (2L) and agitated for 15 minutes. The pH was checked to ensure it was about between 1-2. The organic layer was then separated and checked by chiral HPLC (should be >98%). The organic layer was washed with 1M HCl (2×2L), agitated for 15 minutes and the layers separated. The organics were again washed with 2×2L saturated sodium bicarbonate solution, agitated for 15 minutes, and the layers separated. The organic layer was washed with 2L brine, the phases separated, and the organic layer dried over anhydrous magnesium sulfate. The dried organic layer was filtered and stripped under vacuum to remove solvents and unreacted t-butyl acetate, maintaining a high-vacuum (pump) for at least 20 hours to ensure the removal of t-butyl acetate and siloxanes. A small sample was analyzed using ¹H NMR (CDCl3), TLC (1:1 hexanes/ethyl acetate) and HPLC. Product purity should be close to 90%. If not, this compound can be chromatographed over silica using 20% ethyl acetate/hexanes. In most cases, the compound should be use-tested first in the next step (set forth in Example 6 below) with a 10 g run, before scale-up. Note: If the ¹H NMR still contains imidazole peaks (at 7.00 (s) and 7.62 (s) ppm), rework with 2L MTBE and wash twice with 500 mL 1N HCl, twice with 500 mL saturated NaHCO₃, once with 500 mL brine and dry over MgSO₄. Yield of 14, 220 g (79%). If chemically pure final product is sought, it is recommended that one should not proceed to the next step unless chiral purity of compound 14 exceeds 95%.

### Example 6

Preparation of 15

| Raw material | Source | Amount | | MW | Moles |
|---|---|---|---|---|---|
| 14 | — | 257 | g | 351 | 0.732 |
| Anhydrous THF | Fisher | 4.5 | L | — | — |
| 60% Sodium hydride | Aldrich | 29.2 | g | 24.1 | 0.732 |
| in mineral oil | | | | | |
| 1C (90%) | — | 350 | g | 330.3 | 0.952 |
| 1M HCl | Stock | 1 | L | — | — |
| MTBE | Fisher | 6 | L | — | — |
| Brine | stock | 1.6 | L | — | — |
| Trifluoroacetic acid (TFA) | Aldrich | 210 | mL | — | — |
| Satd. Sodium bicarbonate | Stock | 4 | L | — | — |
| Anhyd. Magnesium sulfate | Fisher | 150 | g | — | — |
| Methylene Chloride | Fisher | 500 | mL | — | — |

Procedure:

Sodium hydride was slurried in 2.5L THF under argon and cooled to −5° C. 14 (257 g) was dissolved in IL THF and added via an addition funnel to sodium hydride over 15 minutes. The solution was stirred for 30 minutes keeping the internal temperature at between 0° C. and 3° C. Compound 1C (340 g) was dissolved in 1L of THF and added via an addition funnel to the solution, keeping the internal temperature between 0° and 5° C. to form a reaction mixture. The reaction mixture about was then slowly warmed to an ambient temperature over 2 hours and held at room temperature for about 20 hours. 1L 1M HCl and 3L MTBE were added and the reaction mixture agitated for 15 minutes. The organic layer was separated, washed twice with 800 mL brine and dried over anhydrous magnesium sulfate. The dried organic layer was then filter over celite and stripped to dryness under vacuum yielding 510 g of intermediate epimers which were taken directly on to the decarboxylation step set forth below.

The intermediate epimers were dissolved in 500 mL methylene chloride to which 210 mL trifluoroacetic acid (TFA) was added before being stirred for between 6-20 hours at ambient temperature. The resulting solution was analyzed by TLC (20% ethyl acetate/hexanes with ceric ammonium sulfate/molybdic acid staining agent). One major spot corresponding to the compound 15 (Rf 0.3) was observed. The solvents were stripped off under vacuum and the concentrated oil dissolved in 2L MTBE. The oil was then washed with saturated sodium bicarbonate solution (4×1L). Stir a minimum of 15 minutes per wash. (Note: For effective removal of TFA, pour MTBE extract into a rapidly stirring aqueous bicarbonate solution.) The organics were then washed with 500 mL of brine, dried over anhydrous magnesium sulfate, filtered over celite and stripped under vacuum. Yield of crude 15 was 492 g. The crude ¹H NMR spectrum run in CDCl₃ indicated a purity level of about 60%.

Crude 15 (492 g) was preabsorbed on silica gel (1 kilo). A column was charged using a 9:1 loading (4 kilos, preferably 15:1) and eluted with 10% EtOAC/Hexanes (2 column volumes), 15% (2 column volumes), 20% (2 column volumes). The compound elutes after 5-6 column volumes. ˜140 g pure 15 was isolated containing a ketone by-product that co-elutes with, the desired 15. The final UV purity was found to be ˜88% with the remaining 12% being the ketone impurity. Note: This ketone impurity was not removed until after the enzymatic ester hydrolysis set forth in Example 9 below. Yield of 15 was 45%.

### Example 7

Preparation of 16:

| Raw material | Source | Amount | | MW | Moles |
|---|---|---|---|---|---|
| 15 | — | 183 | g | 429.4 | 0.364 |
| THF (7 mL/g) | Fisher | 1.3 | L | — | — |
| H₂SO₄ (conc.) | Fisher | 37 | g | 18 M | 0.364 |
| 10% Pd/C (10 wt %) | Aldrich | 18 | g | 10 wt. % | — |
| 16 | — | 143 | g | 393.15 | — |

Procedure:

Crude 15 (183g) was dissolved in 1.3L THF in a 2L hydrogenator flask followed by the addition of concentrated sulfuric acid (1.0 eq., 0.364 mol, 20 mL,) 37 g by weight. The solution was then purged with argon (sub-surface for 15 minutes.) 10% by weight of palladium catalyst (18 g) was charged to the reactor maintaining the argon purge. The flask was then charged with hydrogen, evacuated three times, then stirred under pressure (40 psi) for 5-10 hours, until reaction was complete by HPLC. The reaction was monitored by TLC (50% THF/hexanes, with ceric sulfate, phosphomolybdic acid stain) and HPLC (gluco method). The catalyst was then filtered off through a pad of celite and the solvents stripped under vacuum on a rotary evaporator. Yield of crude 16 was 170 g (120%) as a yellow oil.

### Example 8

Preparation of 17:

| Raw material | Source | Amount | | MW | Moles |
|---|---|---|---|---|---|
| 16 | — | 170 | g | 393.2 | 0.364 |
| | | | crude | | |
| CH₂Cl₂ (ACS) | Fisher | 2.9 | L | — | — |
| DIPEA (2.1 eq.) d = 0.742 | Aldrich | 133 | mL | 129.3 | 0.764 |
| 5-methyl-3-carboxy acid | Maybridge | 58 | g | 145.6 | 0.400 |
| chloride isoxazole 3 | Chem. Co. | | | | |
| (1.1 eq.) | | | | | |
| 1N HCl | Stock | 0.8 | L | — | — |
| Satd. Sodium bicarbonate | Stock | 0.8 | L | — | — |
| Anhydrous. Sodium sulfate | Fisher | 100 | g | — | — |
| 17 | — | 164 | g | 402.18 | — |

Procedure:

In a 5L round bottom flask, the crude 16 (170 g) was dissolved in methylene chloride (2.9L) and cooled to 0° C. (internal temperature) with an ice/salt bath while under a blanket of argon. The yellow solution was charged with isoxazole acid chloride (58 g) as a liquid (thawed at 35° C. in a warm water bath). Due to stability concerns, it is advisable to store the acid chloride cold. Slowly, the diisopropylethyl amine (0.13L) was added via an addition funnel over 10 minutes. The reaction mixture was then allowed to slowly warm to room temperature while monitoring the reaction by TLC and finally by HPLC (generally complete within 1 hour). The reaction was quenched with 1M HCl (400 mL), the aqueous layer removed and the organics reextracted with 1M HCl (400 mL). The aqueous layer was removed and the organics reextracted with saturated bicarbonate (2×400 mL). The organics were dried over sodium sulfate (100 g), filtered and concentrated under vacuum to yield 164 g (112%) of compound 17 as a crude yellow oil.

### Example 9

Preparation of 12:

| Raw material | Source | Amount | | MW | Moles |
|---|---|---|---|---|---|
| 17 | — | 164 | g | 402.2 | 0.364 |
| | | | (crude) | | |
| THF | Fisher | 115 | mL | — | — |
| KH₂PO₄ Buffer | Stock | 12 | L | 0.1 M | — |
| KH₂PO₄ (>99.5%) | Fluka | 163 | g | 136.1 | 1.2 |
| NaOH (10N) | Stock | ˜40 | mL | 40 | — |
| PPL-Type II (crude) | Aldrich | 123 | g | crude | — |
| 0.75 g/g | | | | | |
| HCl (conc.) | Fisher | ˜80 | mL | — | — |
| 12 | — | 142 | g | 390.2 | — |

Procedure:

To make 12L of a buffer, 163 g of potassium dihydrogen phosphate was added to 12L de-ionized water (pH=4-5), and the pH adjusted to between about 7.0-7.2 with 10M NaOH (˜40mL) at a temperature of 37-40° C. 17 (164 g) was dissolved in THF (115 mL) and added to the buffer solution at 37-40° C. The solution may appear biphasic at the onset of the reaction. The PPL enzyme (123 g) was added to the reaction mixture and stirred at 37-40° C. before being quenched with 12M HCl to a pH of between about 1-1.5. The resulting mixture was stirred for 20 minutes while cooling to room temperature. Both the enzyme and the product were caused to crash out into the aqueous phase. The reaction mixture was filtered over a pad of celite collecting the product and enzymes. The pad was dried. The receiver flask was replaced with a clean one, and the top half of the celite pad slurry washed with methylene chloride (3×750 mL). The organics were combined (remove excess water by extraction if present in a large quantity), dried over magnesium sulfate, filtered and concentrated under vacuum to yield a white solid. The white solid was dried under vacuum to yield 150 g of a crude dry product. A column was packed with 1.5 Kg silica slurried in a 4L 1:80:20 (i-PrOH: CH₂Cl₂: Hexanes) mixture. The crude dry product was dry loaded on ˜250 g silica and delivered to the column with ˜500 mL of head space solvent. Two column volumes (˜2×6L) were added of the same eluent mixture then ˜2×6L 3:80:20 (i-PrOH: CH₂Cl₂: Hexanes) were added. Yield of crude 12 was 150 g. The combined yield over three steps after chromatography was between about 65-70%, 94 g (71%).

### Example 9A

Purification of 12

12 was purified via an aqueous sodium bicabonate extraction and subsequent precipitation by acidification. The compound was partially dissolved in 60 volumes of saturated sodium bicarbonate and extracted with 10 volumes of methyl test-butyl ether. The resulting organic layer was extracted twice with 10 volumes of saturated sodium bicarbonate solution. The aqueous bicarbonate extracts were then combined and acidified to a pH of about 1. The resulting offgassing of carbon dioxide maintained the solution at a temperature of above about 20° C. The product, AG7172, precipitated out of the solution upon the acidification. The 12 was then filtered off, washed with 4 volumes of water, and dried in vacuuo at 50° C. with a nitrogen purge.

### Example 10

Procedure for the preparation of compound 6: (See scheme 7 for the structures of the compounds referred to in Examples 10 & 11)

Procedure:

36.80 g (200 mL) of compound 1A was dissolved in acetone (400 mL) before 22.26 g (220mmol) triethylamine was slowly added, keeping the temperature below 30° C. 37.6 g (220mmol) benzyl bromide was then added to form a reaction mixture which was stirred for about 65 hours at which time HPLC showed completion. 200 mL of MTBE was added to the reaction mixture followed by minutes of stirring before the mixture was filtered through a short pad of silica gel to remove most of the precipitated triethylamine salt. The silica gel was then washed with MTBE (200 mL) and the filtrates combined. The filtrates were then washed with 1M HCl (200 mL), saturated sodium bicarbonate (100 mL×2) and brine (200 mL). They were then dried with magnesium sulfate, filter through a short pad of silica gel and concentrated to yield the compound 6 (yields 71-75%; 35.5-37.5 g; having a HPLC purity of between 90->95%). The compound 6 may then be recrystalized in hexanes/methylene chloride (8:1) to yield a crystalline product.

### Example 11

Preparation of compound 7 from compound 6.

Procedure:

1.37 g (5 mmol) of compound 6 was dissolved in 40 mL methylene chloride and cooled to −10° C. 0.93 mL Tf₂O (5.25 mmol) was then added to the solution followed by the slow addition of 0.64 mL (5.25 mmol) 2,6-lutidine. Because the reaction was quite exothermic the temperature was maintained under −8° C. using a cooling bath. After removal from the cooling bath and stirring the reaction mixture for about 1 hour allowing the mixture to warm, the resulting mixture was concentrated under house vacuum. The resulting crude oil was then dissolved in hexanes (100 mL) and stirred over dry ice to precipitate out a pink solid, a lutidinium salt. The precipitate was filtered through a thin layer of silica gel and concentrated again to yield a colorless oil, compound 7. The yield was 90% (1.84 g), which was found to be pure by ¹H NMR.

### Example 12

Procedure for the preparation of compound 18 from Z-Valine. (See scheme 8 for the structures of the compounds referred to in Examples 12-15).

Procedure:

50.26 g (200 mmol) Z-valine followed by 35.0 g (210 mmol) 1,1′-carbonyldiimidazole were dissolved in THF (200 mL) at room temperature. The resulting mixture was stirred for 1 hour at room temperature to yield an acyl imidazole intermediate in solution. (Note: the reaction releases carbon dioxide.) In a separate contained, LiHMDS (1M in THF, 642 mL) was added to THF (800 mL) at −78° C. followed by slow addition of o-benzyl acetate (30 g, 200 mmol). Because the reaction is exothermic the temperature kept at under −70° C. This mixture was stirred for 30 minutes before the acyl imidazole solution was added to the mixture slowly and at a temperature under −68° C. This reaction is also very exothermic, thus, the temperature was maintained under −68° C. The resulting reaction mixture was stirred for 55 minutes, then remove from the dry ice bath. 1M HCl (500 mL) was slowly added to the reaction mixture keeping the temperature under 25° C. The organic layers were then separated, washed with saturated sodium bicarbonate (200 mL) and brine (200 mL), dried with magnesium sulfate and concentrated to yield compound 18 at a yield >85% (>72.09 g) and having a HPLC purity of between 90-95%. To prevent decomposition, compound 18 was kept in a fridge.

### Example 13

Procedure for the preparation of 19 from 18.

Procedure:

A solution of compound 18 (1.38 g, 3.60 mmol) in THF (10 mL) was added slowly to a solution of NaH (60%, 158.4 mg, 3.96 mmol) in THF (20 mL) at −10° C. The resulting reaction mixture was removed from the cooling bath and allowed to warm while being stirred for 20 minutes. To the reaction mixture was then added a solution of compound 7 (1.76 g, 4.33 mmol) in methylene chloride (10 mL). The progress of the reaction was monitored using HPLC to observe the disappearance of the starting materials. The reaction mixture was then stirred for 48 hours before MTBE (50 mL) was added. After 1M HCl (75 mL) was slowly added, the reaction mixture was separated and the aqueous layer extracted by MTBE (50 mL×2). The combined organic layer was then dried in magnesium sulfate, filtered through a short pad of silica gel and concentrated to yield a crude product of compound 19. (Calc. MS 639, found: MH⁺640 and MNa⁺662). After chromatographic separation using hexanes/ethyl acetate (4:1) this process produced yields of 50-60%.

### Example 14

Preparation of compound 20 from compound 19.

Procedure:

Compound 19 (680 mg, 1.06 mmol) was dissolved in a degassed mixture of THF (10 mg) and concentrated sulfuric acid (116 mg, 1.10 mmol). To that solution was added 10% Pd-C (204 mg) before the resulting reaction mixture was stirred in a Parr shaker under a pressure of 50 psi for 5 hours. The mixture was then dissolved in methanol (75 mL), flittered through celite and the celite washed through with methanol (75 mL) to yield 480 mg of a crude material of compound 20 (yield quantitative by weight with overall yields 50-60% for the two-step sequence from compound 18), which issued for the next process without further purification.

### Example 15

Procedure for the preparation of 12 from compound 20.

Procedure:

Compound 20 (300 mg, 0.813 mmol) followed by diisopropylethylamine (DIEPA) (0.45 mL, 2.60 mmol, 3.2 eq) were dissolved in dioxane (40 mL) to give a suspension at 0° C. To that solution was added a solution of 5-methyl-3-isoxazole-3-carbonyl chloride (130 mg, 0.894 mmol) in dioxane (10 mL) at 0° C. (Note: the reaction is very exothermic.) The reaction was monitored by TLC and the reaction mixture stirred for 1 hour. Methylene chloride (20 mL) was then added before the mixture was washed with 1M HCl (10 mL) and saturated sodium bicarbonate (10 mL), dried with magnesium sulfate and filtered through a short pad of silica gel to yield the compound 12 at yields of between 65-70% having a HPLC purity of 95% (cacl. MS 390, found: MNa⁺413) after column separation (methylene chloride/hexanes/isopropanol=79:20:3).

### Example 16

| Raw material | Equiv. | Mmol | FW | Amount | |
|---|---|---|---|---|---|
| 10 | 1.5 | 1.8 | 326.39 | 751 | mg |
| TFA | 12.0 | 18.0 | 114.02 | 1.4 | mL |
| DCM | 10 mL/g of 10 | — | — | 7.6 | mL |
| n-Methylmorpholine | 10.0 | 15.0 | 101.15 | 1.6 | mL |
| DMF | 7 mL/g of 12 | — | — | 4.1 | mL |
| CDMT | 1.05 | 1.6 | 175.58 | 281 | mg |
| DMF | 4 mL/g of CDMT | — | — | 1.1 | mL |
| Water | 42 mL/g of 12 | — | — | 24.7 | mL |

Preparation of compound 11 from compound 10.

Procedure:

Compound 10 was dissolved in DCM in a 1 neck round bottom flask and cover with a septum. The flask was then purged with nitrogen followed by the addition of TFA via syringe while the solution was being stirred. The progress of the reaction was monitored by TLC using 5% MeOH in DCM until after about 4 hours the starting material disappeared. The solvent and excess TFA were removed under vacuum at pressure of <50 mTorr at 45° C. The product, compound 11, was used immediately in the step set forth below.

Preparation of compound AG7088 from compounds 11 and 12.

Procedure:

Compounds 11 and 12 were dissolved in DMF in a 1 neck flask covered with a septum and fitted with a temperature probe. The flask was purged with nitrogen. The resulting solution was divided into two portions. In a first portion was added n-methylmorpholine via syringe and cooled to 0° C.±5° C. In a second portion of the solution CDMT was dissolved. This CDMT solution was then added dropwise via syringe to the first portion of the solution, maintaining the reaction temperature of 0° C. ±5° C. The resulting reaction mixture was then allowed to warm to room temperature. The reaction was monitored for about 1 hour by TLC (7:3:1 hexanes:EtOAc:IPA) until the compound 12 disappeared. Once the reaction was complete the product AG7088 was precipitated out of solution by the slow addition of water to reaction mixture. The resulting slurry was filtered to obtain a yield of >85% white granular crystals of compound AG7088 having a purity of >97%. The product may then be recrystallized by dissolving it in hot MeOH:EtOAc 1:1 followed by slow addition of hexanes (2 vols.)

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

What is claimed is:

1. A process useful for preparing an antipicornaviral agents of formula IA: 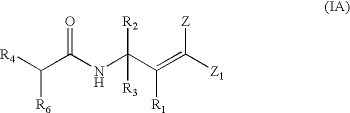
comprising:
Step A: preparing a compound of formula IIA: 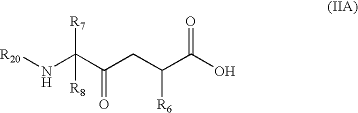
comprising the substeps of:
(a) converting a compound of formula XIII to a β-ketoester of formula XIV by reacting said compound of formula XIII with 1,1′-carbonyldiimidazole followed by treatment with a compound of formula XIIIA; 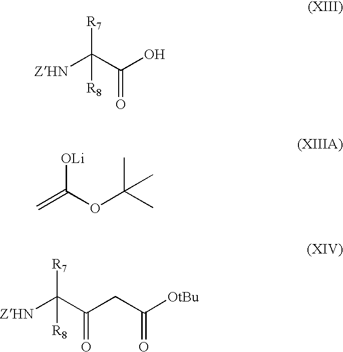
(b) converting the β-ketoester of formula XIV to an enolate of formula XV by reacting said β-ketoester of formula XIV with a compound of formula XVI; 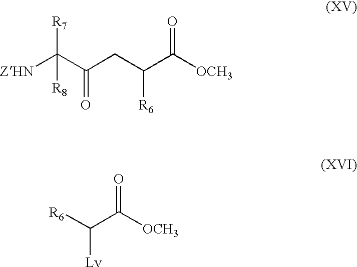
(c) hydrogenolyzing the enolate of formula XV to yield a compound of formula XVII; 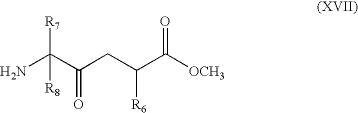
(d) acylating the compound of formula XVII by reacting said compound of formula XVII with a compound of formula R₂₀-X to yield a compound of formula XVIII, wherein X is any suitable halide; and 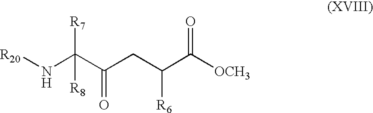
(e) enzymatic hydrolyzing of the compound of formula XIII to yield the compound of formula IIA; and
Step B: subjecting the compound of formula IIA to an amide-forming reaction 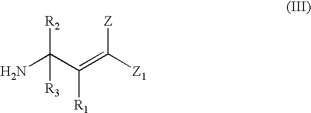
with a compound of formula III:
wherein Lv is any suitable leaving group;
Z′ is any suitable protecting group for an N atom;
R₁ is H, F, an alkyl group, OH, SH, or an O-alkyl group;
R₂ and R₃ are each independently H, 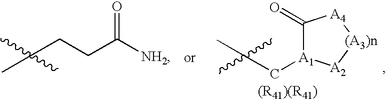
where n is an integer from 0 to 5, A₁ is CH or N, A₂ and each A₃ are independently selected from C(R₄₁)(R₄₁), N(R₄₁), S, S(O), S(O)₂, and O, and A₄ is NH or NR₄₁, where each R₄₁ is independently H or lower alkyl, provided that no more than two heteroatoms occur consecutively in the above-depicted ring formed by A₁, A₂, (A₃)ₙ, A₄ and C═O, and at least one of R₂ and R₃ is 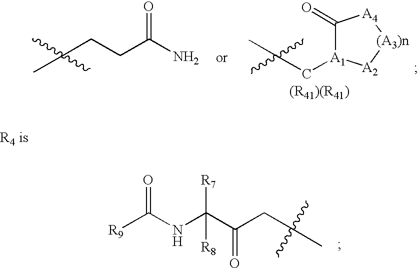
R₆ is H, F, an alkyl group, a cycloalkyl group, a heterocycloalkyl group, an aryl group, or a heteroaryl group;
R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇R₁₈, where R₁₇, R₁₈, and R₁₉ are each independently H, an alkyl group, a cycloalkyl group, a heterocycloalkyl group, an aryl group, a heteroaryl group, or an acyl group, provided that at least one of R₇ and R₈ is an alkyl group, an aryl group, a heteroaryl group, —OR₁₇, —SR₁₇, —NR₁₇R₁₈, —NR₁₉NR₁₇R₁₈, or —NR₁₇OR₁₈;
R₉ is a five-membered heterocycle having from one to three heteroatoms selected from O, N, and S;
R₂₀ is 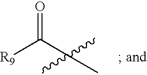
Z and Z₁ are each independently H, F, an alkyl group, a cycloalkyl group, a heterocycloalkyl group, an aryl group, a heteroaryl group, —C(O)R₂₁, —CO₂R₂₁, CN, —C(O)NR₂₁R₂₂, —C(O)NR₂₁OR₂₂, —C(S)R₂₁, —C(S)NR₂₁R₂₂, —NO₂, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), —PO(NR₂₁R₂₂)(NR₂₃R₂₄), —C(O)NR₂₁NR₂₂R₂₃, or —C(S)NR₂₁NR₂₂R₂₃, where R₂₁, R₂₂, R₂₃, and R₂₄ are each independently H, an alkyl group, a cycloalkyl group, a heterocycloalkyl group, an aryl group, a heteroaryl group, an acyl group, or a thioacyl group, or where any of two of R₂₁, R₂₂, R₂₃, and R₂₄, together with the atom(s) to which they are bonded, form a heterocycloalkyl group, provided that Z and Z₁ are not both H;
or Z₁ and R₁, together with the atoms to which they are bonded, form a cycloalkyl or heterocycloalkyl group, where Z₁ and R₁ are as defined above except for moieties that cannot form the cycloalkyl or heterocycloalkyl group;
or Z and Z₁, together with the atoms to which they are bonded, form a cycloalkyl or heterocycloalkyl group, where Z and Z₁ are as defined above except for moieties that cannot form the cycloalkyl or heterocycloalkyl group.

2. A process useful for preparing a compound of formula IIA: 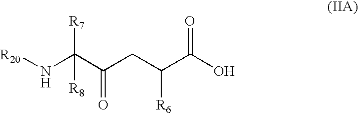
comprising:
(a) converting a compound of formula XIII to a β-ketoester of formula XIV by reacting said compound of formula XIII with 1,1′-carbonyldiimidazole followed by treatment with a compound of formula XIIIA; 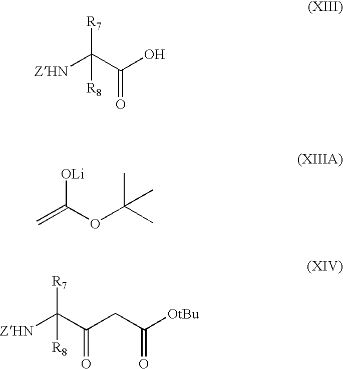
(b) converting the β-ketoester of formula XIV to an enol ate of formula XV by reacting said β-ketoester of formula XIV with a compound of formula XVI; 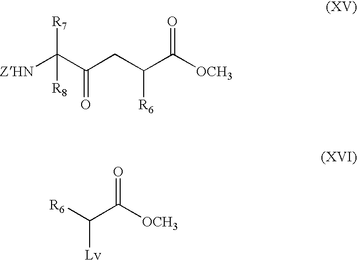
(c) hydrogenolyzing the enolate of formula XV to yield a compound of formula XVII; 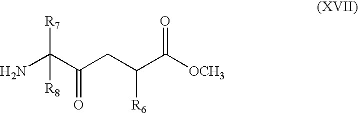
(d) acylating the compound of formula XVII by reacting said compound of formula XVII with a compound of formula R₂₀-X to yield a compound of formula XVIII, 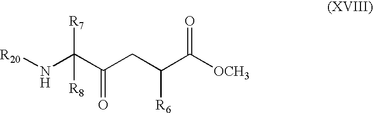
wherein X is any suitable halide; and
(e) enzymatic hydrolyzing of the compound of formula XIII to yield the compound of formula IIA;
wherein Lv is any suitable leaving group;
Z′ is any suitable protecting group for an N atom;
R₆ is H, F, an alkyl group, a cycloalkyl group, a heterocycloalkyl group, an aryl group, or a heteroaryl group;
R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈, where R₁₇, R₁₈, and R₁₉ are each independently H, an alkyl group, a cycloalkyl group, a heterocycloalkyl group, an aryl group, a heteroaryl group, or an acyl group, provided that at least one of R₇ and R₈ is an alkyl group, an aryl group, a heteroaryl group, —OR₁₇, —SR₁₇, —NR₁₇R₁₈, —NR₁₉NR₁₇R₁₈, or —NR₁₇OR₁₈;
R₉ is a five-membered heterocycle having from one to three heteroatoms selected from O,N, and S; and
R₂₀ is

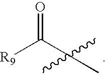

3. The process according to claim 2, wherein Porcine Pancrease Lipase is used as an enzyme in the enzymatic hydrolyzing step.

4. The process according to claim 2, wherein the compound of formula XIII is Z-Valine.

5. The process according to claim 2, wherein the compound of formula XVI is:

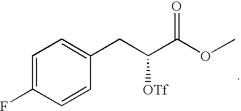

6. The process according to claim 2, wherein the β-ketoester of formula XIV is first reacted with an alkali-metal hydride before reacting the β-ketoester with the compound of formula XVI.

7. The process according to claim 2, wherein the alkali-metal hydride is sodium hydride.

8. The process according to claim 2, wherein step (c) comprises a palladium hydrogenolysis.

9. The process according to claim 8, wherein the palladium hydrogenolysis is carried out under pressure.

10. The process according to claim 2, wherein diisopropylethyl amine is used as a reagent in the acylation step (d).

11. The process according to claim 2, wherein the compound of formula IIA is: 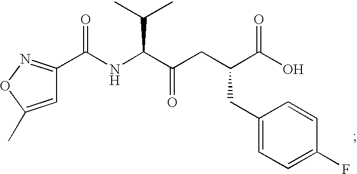
the compound of formula XIII is: 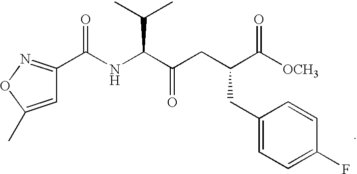
the compound of formula XVII is: 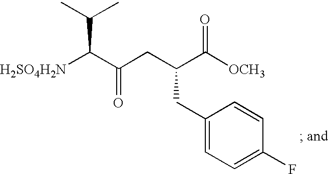
the enolate of formula XV is:

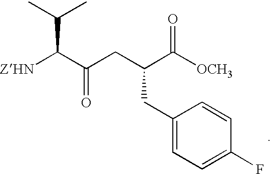

12. A process useful for preparing a compound of formula IIA: 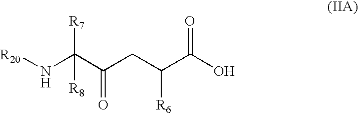
comprising the steps of:
(a) converting a compound of formula XIX to a β-ketoester of formula XX by reacting said compound of formula XIX with 1,1′-carbonyldiimidazole followed by treatment with a compound of formula XIXA; 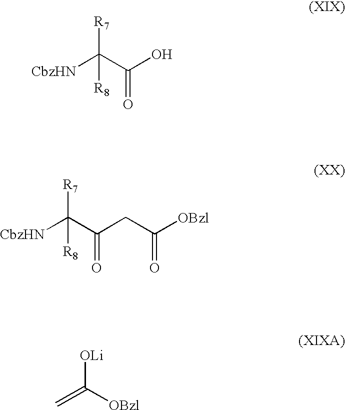
(b) converting the compound of formula XX to a compound of formula XXI by reacting said compound of formula XX with a compound of formula XXII under suitable reaction conditions; 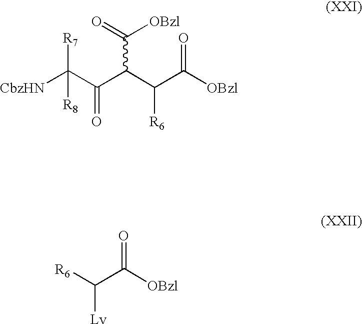
(c) hydrogenating the compound of formula XXI to yield a compound of formula XXIII; and 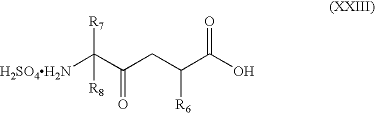
(d) acylating the compound of formula XXIII by reacting it with R₂₀-X under suitable conditions to yield the compound of formula IIA;
wherein X is any suitable halide;
wherein Lv is any suitable leaving group;
Z′ is any suitable protecting group for an N atom;
R₆ is H, F, an alkyl group, a cycloalkyl group, a heterocycloalkyl group, an aryl group, or a heteroaryl group;
R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR₁₇OR₁₈, where R₁₇, R₁₈, and R₁₉ are each independently H, an alkyl group, a cycloalkyl group, a heterocycloalkyl group, an aryl group, a heteroaryl group, or an acyl group, provided that at least one of R₇ and R₈ is an alkyl group, an aryl group, a heteroaryl group, —OR₁₇, —SR₁₇, —NR₁₇R₁₈, —NR₁₉NR₁₇R₁₈, or —NR₁₇OR₁₈; R₉ is a five-membered heterocycle having from one to three heteroatoms selected from O, N, and S; and
R₂₀ is

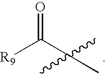

13. The process according to claim 12, wherein the compound of formula XIX is:

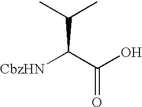

14. The process according to claim 12, wherein the compound of formula XX is:

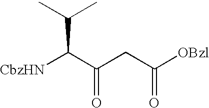

15. The process according to claim 12, wherein the compound of formula XXI is:

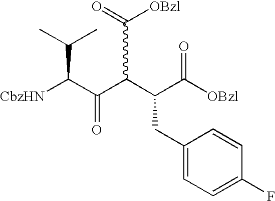

16. The process according to claim 12, wherein the compound of formula XXII is:

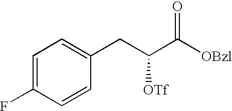

17. The process according to claim 12, wherein the compound of formula XXIII is :

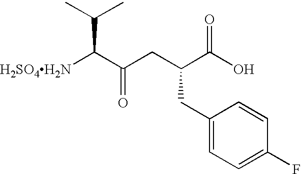

18. A process useful for preparing a compound of formula I comprising the steps of: 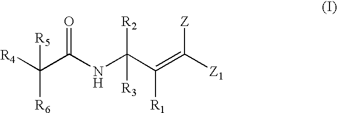
(a) reacting a compound of formula II with a compound of formula IIIA in the presence of N-methylmorpholine to form a reaction mixture; 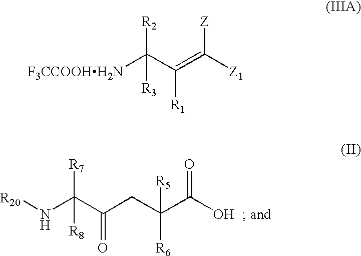
(b) adding a compound of formula Lv-X to the reaction mixture to form a compound of formula I,
wherein X is any suitable halide;
Lv is any suitable leaving group;
R₁ is H, F, an alkyl group, OH, SH, or an O-alkyl group;
R₂ and R₃ are each independently H; 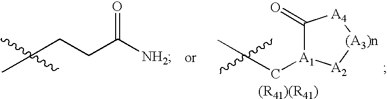
where n is an integer from 0 to 5, A₁ is CH or N, A₂ and each A₃ are independently selected from C(R₄₁)(R₄₁), N(R₄₁), S, S(O), S(O)_{2,} and O, and A₄ is NH or NR₄₁, where each R₄₁ is independently H or lower alkyl, provided that no more than two heteroatoms occur consecutively in the above-depicted ring formed by A₁, A₂, (A₃)ₙ, A₄ and C═O, and at least one of R₂ and R₃ is 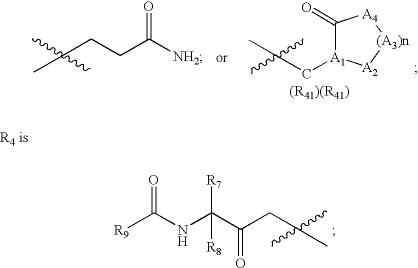
R₅ and R₆ are each independently H, F, an alkyl group, a cycloalkyl group, a heterocycloalkyl group, an aryl group, or a heteroaryl group;
R₇ and R₈ are each independently H, an alkyl group, a cycloalkyl group, a heterocycloalkyl group, an aryl group, a heteroaryl group, —OR₁₇, —SR₁₇, —NR₁₇R₁₈, —NR₁₉NR₁₇R₁₈, or —NR17OR₁₈, where R₁₇, R₁₈, and R₁₉ are each independently H, an alkyl group, a cycloalkyl group, a heterocycloalkyl group, an aryl group, a heteroaryl group, or an acyl group, provided that at least one of R₇ and R₈ is an alkyl group, an aryl group, a heteroaryl group, —OR₁₇, —SR₁₇, —NR₁₇R₁₈, —NR₁₉NR₁₇R₁₈, or —NR₁₇OR₁₈;
R₉ is a five-membered heterocycle having from one to three heteroatoms selected from O, N, and S;
R₂₀ is 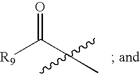
Z and Z₁ are each independently H, F, an alkyl group, a cycloalkyl group, a heterocycloalkyl group, an aryl group, a heteroaryl group, —C(O)R₂₁, —CO₂R₂₁, CN, —C(O)NR₂₁R₂₂, —C(O)NR₂₁OR₂₂, —C(S)R₂₁, —C(S)NR₂₁R₂₂, —NO₂, —SOR₂₁, —SO₂R₂₁, —SO₂NR₂₁R₂₂, —SO(NR₂₁)(OR₂₂), —SONR₂₁, —SO₃R₂₁, —PO(OR₂₁)₂, —PO(R₂₁)(R₂₂), —PO(NR₂₁R₂₂)(OR₂₃), —PO(NR₂₁R₂₂)(NR₂₃R₂₄), —C(O)NR₂₁NR₂₂R₂₃, or —C(S)NR₂,NR₂₂R₂₃, where R₂₁, R₂₂, R₂₃, and R₂₄ are each independently H, an alkyl group, a cycloalkyl group, a heterocycloalkyl group, an aryl group, a heteroaryl group, an acyl group, or a thioacyl group, or where any of two of R₂₁, R₂₂, R₂₃, and R₂₄, together with the atom(s) to which they are bonded, form a heterocycloalkyl group, provided that Z and Z₁ are not both H;
or Z₁ and R₁, together with the atoms to which they are bonded, form a cycloalkyl or heterocycloalkyl group, where Z₁ and R₁ are as defined above except for moieties that cannot form the cycloalkyl or heterocycloalkyl group;
or Z and Z₁, together with the atoms to which they are bonded, form a cycloalkyl or heterocycloalkyl group, where Z and Z₁ are as defined above except for moieties that cannot form the cycloalkyl or heterocycloalkyl group.

19. The process according to claim 18, wherein the compound of formula I is:

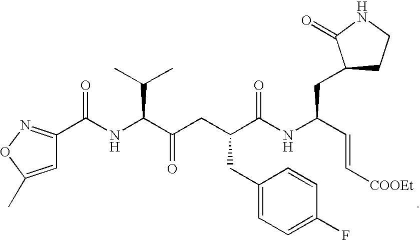

20. The process according to claim 18, wherein the compound of formula II is:

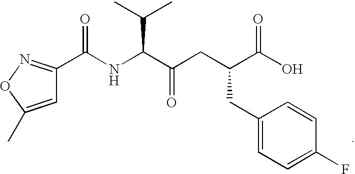

21. The process according to claim 18, wherein the compound of formula IIIA is:

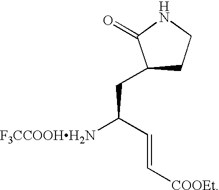

22. The process according to claim 18, wherein the compound of formula Lv-X is chlorodimethyltriazine.

23. The process according to claim 18, wherein the compound of formula IIIA is prepared by a process comprising the step of reacting a compound of formula IIIB with trifluoroacetic acid, wherein the compound of formula IIIB is:

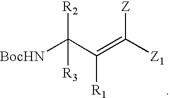

24. A process useful for preparing a compound of formula XVIA: 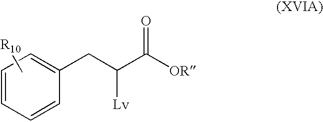
wherein R₁₀ is a halogen or an alkyl group; comprising:
Step A: converting a compound of formula VI to a compound of formula V comprising the substeps of:
(a) reacting a R₁₀ substituted benzaldehyde of formula VI: 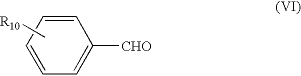
with hydantoin in an aqueous medium in the presence of a catalyst at reflux temperature to form a reaction mixture;
(b) treating the reaction mixture with an excess of an alkali metal hydroxide at reflux temperature to form a alkali metal hydroxide-treated solution;
(c) adding an alkali metal halide to the alkali metal hydroxide-treated solution to give a solution;
(d) acidifying the solution with a concentrated acid to give a precipitate of formula V; 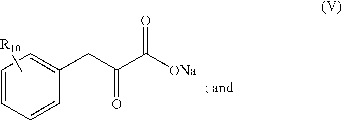
Step B: performing an enzymatic reduction of the compound of formula V to a compound of formula VII; 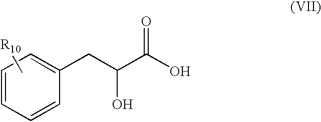
Step C: esterifying the compound of formula VII to a compound of formula XII by reacting the compound of formula VII with a compound of formula R″—OH, wherein R″ is an alkyl or aryl; 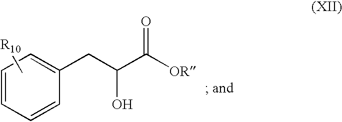
Step D: converting the compound of formula XII to the compound of formula XVIA.

25. The process according to claim 24, wherein the reduction reaction of step B is catalyzed by formate dehydrogenase and lactate dehydrogenase.

26. The process according to claim 24, wherein the reduction reaction of step B uses membrane-enclosed enzymatic catalysis.

27. The process according to claim 24, wherein the reduction reaction of step B uses coimmobilization enzymatic catalysis.

28. The process according to claim 27, wherein the coimmobilization enzymatic catalysis uses PAN 500 as a suitable copolymer.

29. The process according to claim 25, wherein the lactate dehydrogenase is D-lactate dehydrogenase.

30. The process according to claim 25, wherein the lactate dehydrogenase is L-lactate dehydrogenase.

31. The process according to claim 24, wherein the esterification step C is performed at about room temperature in the presence of hydrochloric acid and dioxane.

32. The process according to claim 24, wherein the catalyst used in step (a) is primary or secondary amine.

33. The process according to claim 32, wherein the catalyst is 1-amino-2-propanol.

34. A process for preparing a compound of formula XVIA: 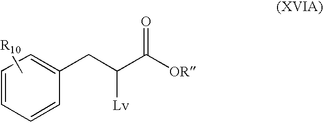
comprising:
Step A′ converting serine to a compound of formula VII: 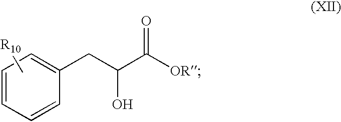
comprising the substeps of:
(a) converting serine to potassium glycidate by a standard process; and
(b) carrying out a regioselective epoxide ring-opening reaction with a compound of formula R₁₀-phenyl-Q;
Step B′: esterifying the compound of formula VII to a compound of formula XII by reacting the compound of formula VII with a compound of formula R″—OH; 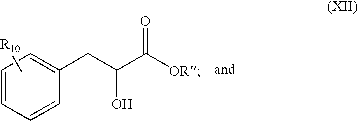
Step C′: converting the compound of formula XII to the compound of formula XVIA;
wherein R₁₀ is a halogen or an alkyl group; R″ is an alkyl or aryl; and
Q is an activated bromide, a sulfate, or a primary iodide.

35. The process according to claim 34, wherein the serine is L-serine.

36. The process according to claim 34, wherein the serine is D-serine.

37. The process according to claim 34, wherein Q is —MgBr.

38. The process according to claim 34, wherein R₁₀ is F in the para-possition of the phenyl ring.

39. The process according to claim 34, wherein the esterification step B′ is performed at about room temperature in the presence of hydrochloric acid and dioxane.

40. The process according to claim 34, wherein the potassium glycidate formed from step A′ (a) is converted to a glycidic acid before the regioselective epoxide ring-opening reaction of step A′ (b) is performed.

41. A process for preparing a compound of formula XVIB: 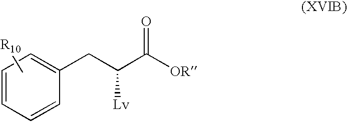
comprising:
Step A″: preparing a compound of formula XIIA from a compound of formula IX: 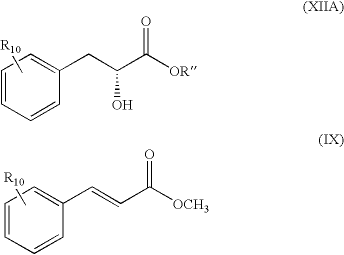
comprising the substeps of:
(a) performing an asymmetric dihydroxylation of a compound of formula IX to form a compound of formula XA: 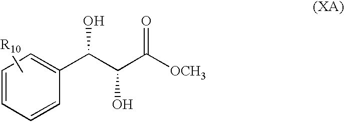
(b) reacting the compound of formula IX with 1,1′-carbonyldiimidazole in the presence of toluene to form a compound of formula XI; and 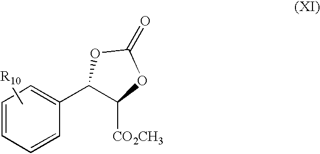
(c) performing a palladium-mediated reduction of the compound of formula XI to form the compound of formula XIIA; and
Step B″ the conversion of the compound of formula XIIA to the compound of formula XVIB;
wherein R₁₀ is a halogen or an alkyl group; and
R″ is an alkyl or aryl.

42. The process according to claim 41, wherein the asymmetric dihydroxylation is a Sharpless asymmetric dihydroxylation.

43. The process according to claim 41, wherein step (b) is performed at about 80° C.

44. The process according to claim 41, wherein the palladium-mediated reduction step is done in the presence of formic acid at about room temperature.

45. A compound of formula IVA: 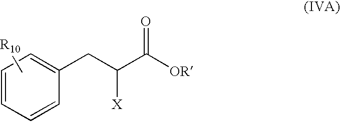
wherein, R₁₀ is a halogen or alkyl group;
X is OH, OSO₂CF₃, OSO₂CH₃, OSO₂(p-tolyl), halide or any other leaving group; and
R′ is H, alkyl or al group.

46. The compound according to claim 45, wherein R₁₀ is F.

47. The compound according to claim 46, wherein F is substituted at the para position of the phenyl ring.

48. The compound according to claim 45, wherein X is OH.

49. The compound according to claim 45, wherein R′ is methyl.

50. A compound of t he following formula: 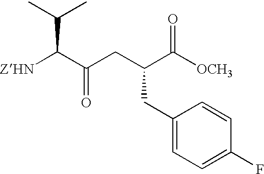
and acid addition salts thereof.

51. A compound of the following formula: 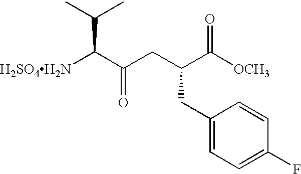
and acid addition salts thereof.

52. A compound of the following formula: 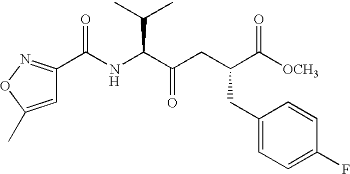
and acid addition salts thereof.

53. A compound of the following formula: 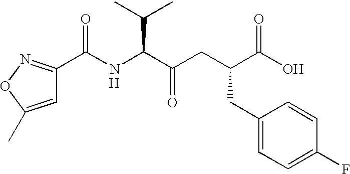
and acid addition salts thereof.

54. A process for preparing a compound of formula VII: 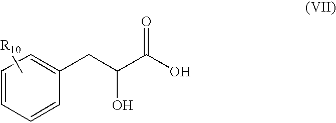
wherein R₁₀ is a halogen or an alkyl group;
comprising:
Step A: converting a compound of formula VI to a compound of formula V comprising the substeps of:
(a) reacting a R₁₀ substituted benzaldehyde of formula VI: 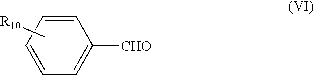
with hydantoin in an aqueous medium in the presence of a catalyst at reflux temperature to form a reaction mixture;
(b) treating the reaction mixture with an excess of an alkali metal hydroxide at reflux temperature to form a alkali metal hydroxide-treated solution;
(c) adding an alkali metal halide to the alkali metal hydroxide-treated solution to give a solution;
(d) acidifying the solution with a concentrated acid to give a precipitate of formula V: 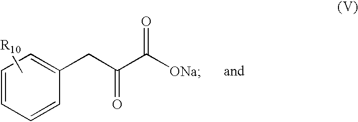
Step B: the enzymatic reduction of the compound of formula V to the compound of formula VII.

55. A process for preparing a compound of formula VII 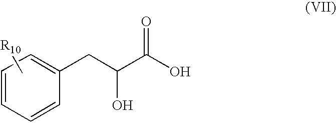
comprising the steps:
(a) converting serine to potassium glycidate by a standard process;
(b) carrying out a regioselective epoxide ring-opening reaction with a compound of formula R₁₀-phenyl-Q to yield the compound of formula VII, wherein R₁₀ is a halogen or an alkyl group; and
Q is an activated bromide, a sulfate, or a primary iodide.

56. The process according to claim 55, wherein the potassium glycidate formed from step (a) is converted to a glycidic acid before the regioselective epoxide ring-opening reaction of step (b) is performed.

57. A process for performing a large-catalyst catalyzed reaction comprising:
(a) placing a reagent and a large catalyst in a continuous membrane reactor having a reactor volume;
(b) allowing the large catalyst catalyzed reaction to occur in the continuous membrane reactor; and
(c) collecting a product from the continuous membrane reactor, wherein the continuous membrane reactor comprises a tangential flow filter unit, a reactor loop to circulate the reagent and the large-catalyst through the tangential flow filter unit, and a substrate feed pump for feeding the reagent into the reactor loop, wherein the reactor loop has a reactor loop volume and comprises:
(i) a tube; and
(ii) a circulation pump.

58. The process as claimed in claim 57, wherein the large-catalyst is an enzyme.

59. The process as claimed in claim 57, wherein the large-catalyst is an anchored catalyst.

60. The process as claimed in claim 57, wherein the large-catalyst has a molecular volume larger than the molecular volume of the product.

61. The process as claimed in claim 57, wherein the reactor loop volume is at least 50% of the reactor volume.

62. The process as claimed in claim 57, wherein the reactor loop volume is at least 80% of the reactor volume.

63. The process as claimed in claim 57, wherein the reactor loop volume is at least 95% of the reactor volume.
